(19) United States
(12) Patent Application Publication
(10) Pub. No.: US 2005/0222252 A1
(43) Pub. Date: Oct. 6, 2005

(54) Use of cis-Epoxyeicosantrienoic acids and inhibitors of soluble epoxide hydrolase to reduce pulmonary infiltration by neutrophils

(75) Inventors: Bruce D. Hammock, Davis, CA (US); Kent E. Pinkerton, Davis, CA (US); Kevin R. Smith, Davis, CA (US); Takaho Watanabe, Davis, CA (US); Seung Jin Ma, Davis, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: The Regents of the University of California, Oakland, CA (US)

(21) Appl. No.: 10/815,425

(22) Filed: Mar. 31, 2004

(51) Int. Cl. A61K 31/335

(52) U.S. Cl. 514/475

(57) ABSTRACT

It has now been discovered that inhibitors of soluble epoxide hydrolase (“sEH”) are useful in reducing the severity of or inhibiting the progression of obstructive pulmonary diseases, restrictive airway diseases, and asthma. Administering a cis-epoxyeicosantrienoic acid (“EET”) in addition to the inhibitor is at least additive, and may be synergistic, in reducing or inhibiting these conditions and diseases, as measured by reduced numbers of neutrophils present in the lung. The inhibitor of sEH may be a nucleic acid, such as a small interfering RNA.

### STATEMENT AS TO RIGHTS TO INVENTIONS MADE UNDER FEDERALLY SPONSORED RESEARCH AND DEVELOPMENT

This invention was made with government support under grant nos. ES02710 and ES04699 awarded by the National Institutes of Health. The government has certain rights in the invention.

### CROSS-REFERENCES TO RELATED APPLICATIONS

Not Applicable

### REFERENCE TO A “SEQUENCE LISTING,” A TABLE, OR A COMPUTER PROGRAM LISTING APPENDIX SUBMITTED ON A COMPACT DISK.

Not Applicable

### BACKGROUND OF THE INVENTION

Obstructive airway diseases, including emphysema and chronic bronchitis are involved in 26% of smoking-attributable deaths (Peto, R. et al., Lancet 339:1268-1278 (1992)). Chronic obstructive pulmonary disease (COPD) is prevalent in approximately 20 million men and women in the United States and is the fourth leading cause of death with a mortality rate of 20/100,000 (Snider, G. L. ed. Leff, A. R. (McGraw-Hill, New York), pp. 821-828 (1996)). The most common cause of COPD is cigarette smoking. However, quitting smoking does not appear to resolve many of the features of COPD, including the inflammatory response present in the airways (Turato, G. et al., Am J Respir Crit Care Med 152:1262-126 (1995); Rutgers, S. R. et al., Thorax 55:12-18 (2000)). Chronic bronchial inflammation is a common feature involved in the pathogenesis of many diseases such as asthma, acute respiratory distress syndrome, and COPD. The pathology of chronic bronchitis and COPD includes airway mucus gland hyperplasia, mucous hypersecretion, and an influx of inflammatory cells including neutrophils, macrophages, and lymphocytes (Jeffery, P. K. Thorax 53:129-136 (1998); Fournier, M. et al., Am. Rev. Respir. Dis. 140:737-742 (1989); Saetta, M. et al., Am. J. Respir. Crit. Care Med. 156:1633-1639 (1997); Grashoff, W. F. et al., Am. J. Pathol. 151:1785-1790(1997); Saetta, M. et al., Am. J. Respir. Crit. Care Med. 157:822-826 (1998)). Chronic inflammation may also provide the ideal environment for cellular changes that lead to cancer.

Epoxide hydrolases (EHs) are enzymes that add water to epoxides resulting in their corresponding 1,2-diols (Hammock, B. D. et al., in Comprehensive Toxicology: Biotransformation (Elsevier, New York), pp. 283-305 (1997); Oesch, F. Xenobiotica 3:305-340 (1972)). Four principal EH's are known: leukotriene epoxide hydrolase, cholesterol epoxide hydrolase, microsomal EH (“mEH”), and soluble EH (“sEH,” previously called cytosolic EH). The leukotriene EH acts on leukotriene A₄, whereas the cholesterol EH hydrates compounds related to the 5,6-epoxide of cholesterol (Nashed, N. T., et al., Arch. Biochem. Biophysics., 241:149-162, 1985; Finley, B. and B. D. Hammock, Biochem. Pharmacol., 37:3169-3175,1988). The microsomal epoxide hydrolase metabolizes monosubstituted, 1,1-disubstituted, cis-1,2-disubstituted epoxides and epoxides on cyclic systems epoxides to their corresponding diols. Because of its broad substrate specificity, this enzyme is thought to play a significant role in ameliorating epoxide toxicity. Reactions of detoxification typically decrease the hydrophobicity of a compound, resulting in a more polar and thereby excretable substance.

Soluble EH is only very distantly related to mEH and hydrates a wide range of epoxides not on cyclic systems. In contrast to the role played in the degradation of potential toxic epoxides by mSH, sEH is believed to play a role in the formation or degradation of endogenous chemical mediators. For instance, cytochrome P450 epoxygenase catalyzes NADPH-dependent enatioselective epoxidation of arachidonic acid to four optically active cis-epoxyeicosantrienoic acids (“EETs”) (Karara, A., et al., J. Biol. Chem., 264:19822-19877, (1989)). Soluble epoxide hydrolase has been shown in vivo to convert these compounds with regio- and enantiofacial specificity to the corresponding vic-dihydroxyeicosatrienoic acids (“DHETs”). Both liver and lung cytosolic fraction hydrolyze 14,15-EET, 8,9-EET and 11,12-EET, in that order of preference. The 5,6 EET is hydrolyzed more slowly. Purified sEH selects 8S,9R- and 14R,15S-EET over their enantiomers as substrates. Studies have revealed that EETs and their corresponding DHETs exhibit a wide range of biological activities. Some of these activities include involvements in luteinizing hormone-releasing hormone, stimulation of luteinizing hormone release, inhibition of Na⁺/K⁺ATPase, vasodilation of coronary artery, mobilization of Ca^{2 +}and inhibition of platelet aggregation.

### BRIEF SUMMARY OF THE INVENTION

This invention provides a number of uses, compositions, and methods. In one group of embodiments, the invention provides uses for a cis-epoxyeicosantrienoic acid (“EET”) for the manufacture of a medicament to inhibit or slow progression of a condition selected from the group consisting of an obstructive pulmonary disease, an interstitial lung disease, and asthma. The obstructive pulmonary disease can be selected from the group consisting of chronic obstructive pulmonary disease (“COPD”), emphysema, and chronic bronchitis. In some embodiments, the interstitial lung disease is idiopathic pulmonary fibrosis. In other embodiments the interstitial lung disease is one associated with occupational exposure to a dust. In some embodiments, the condition is asthma. The EET can be 14,15-EET, 8,9-EET and 11,12-EET. 5,6-EET is unstable, but may be suitable for some applications. In some embodiments, the EET is 14R,15S-EET. The EET can be in a material which releases the EET into the surrounding environment over time.

In another set of embodiments, the invention provides uses of an inhibitor of soluble epoxide hydrolase (“sEH”) for the manufacture of a medicament to inhibit or slow progression a condition selected from the group consisting of an obstructive pulmonary disease, an interstitial lung disease, and asthma. The obstructive pulmonary disease can be, for example, selected from the group consisting of chronic obstructive pulmonary disease (“COPD”), emphysema, and chronic bronchitis. The interstitial lung disease can be, for example, idiopathic pulmonary fibrosis, or one associated with occupational exposure to a dust. The condition can be asthma. The inhibitor of sEH can be an adamantyl dodecyl urea (such as the butyl ester), a N-cyclohexyl-N′-dodecyl urea (CDU) and a N,N′-dicyclohexylurea (DCU). The medicament can be a slow release formulation. The medicament can further comprise a cis-epoxyeicosantrienoic acid (“EET”). The EET can be 14,15-EET, 8,9-EET, or 11,12-EET. In some preferred embodiments, the EET is 14R,15S-EET.

The invention further provides for the use of a nucleic acid that inhibits expression of soluble epoxide hydrolase (“sEH”) for the manufacture of a medicament for inhibiting or slowing progression of a condition selected from the group consisting of an obstructive pulmonary disease, an interstitial lung disease, and asthma. In some preferred embodiments, the nucleic acid is a small interfering RNA. The obstructive pulmonary disease can be, for example, chronic obstructive pulmonary disease (“COPD”), emphysema, or chronic bronchitis. The interstitial lung disease can be, for example, idiopathic pulmonary fibrosis, or one associated with occupational exposure to a dust. The condition can be asthma.

In yet a further group of embodiments, the invention provides methods of inhibiting progression of a condition selected from the group consisting of an obstructive pulmonary disease, an interstitial lung disease, and asthma. The method comprises administering an inhibitor of soluble epoxide hydrolase (“sEH”) and a cis-epoxyeicosantrienoic acid (“EET”) to a person in need thereof. The obstructive pulmonary disease can be, for example, chronic obstructive pulmonary disease (“COPD”), emphysema, and chronic bronchitis. The interstitial lung disease can be, for example, idiopathic pulmonary fibrosis, or one associated with occupational exposure to a dust. The condition can be asthma. The inhibitor of sEH or the EET, or both, can be in a material which releases the inhibitor over time. The EET can be 14,15-EET, 8,9-EET, or 11,12-EET. In some preferred embodiments, the EET is 14R,15S-EET. The inhibitor can be administered orally. Typically, the inhibitor is administered in a total daily dose from about 0.001 mg/kg to about 100 mg/kg body weight.

In another group of embodiments, the invention provides methods of inhibiting progression of a condition selected from the group consisting of an obstructive pulmonary disease, an interstitial lung disease, and asthma. The methods comprise administering to a person in need thereof a nucleic acid which inhibits expression of a gene encoding soluble epoxide hydrolase (“sEH”), and a cis-epoxyeicosantrienoic acid (“EET”). The obstructive pulmonary disease can be, for example, chronic obstructive pulmonary disease (“COPD”), emphysema, and chronic bronchitis. The interstitial lung disease can be, for example, idiopathic pulmonary fibrosis or one associated with occupational exposure to a dust. The condition can be asthma. The nucleic acid can be a small interfering RNA (“siRNA”).

### BRIEF DESCRIPTION OF THE DRAWINGS

FIG. 1. Blood concentration-time profiles of AUDA-NBE and AUDA in SH rats following subcutaneous administration.

FIG. 2. Number of cells in BAL from rats exposed to tobacco smoke for 3 days. Rats were exposed to filtered air (grey bars) after treatment with vehicle, sEH inhibitor, or sEH inhibitor+EETs. Additional rats were exposed to tobacco smoke (black bars) after treatment with vehicle, sEH inhibitor, or sEH inhibitor+EETs. Data are presented as mean+SE (n=4). a p<0.05, compared to respective filtered air control. b p<0.05, compared to tobacco smoke+vehicle. c p<0.05, compared to tobacco smoke+sEH inhibitor.

FIG. 3. Number of macrophages in BAL from rats exposed to tobacco smoke for 3 days. Rats were exposed to filtered air (grey bars) after treatment with vehicle, sEH inhibitor, or sEH inhibitor+EETs. Additional rats were exposed to tobacco smoke (black bars) after treatment with vehicle, sEH inhibitor, or sEH inhibitor+EETs. Data are presented as mean+SE (n=4). a p<0.05, compared to respective filtered air control. b p<0.05, compared to tobacco smoke+vehicle. c p<0.05, compared to tobacco smoke+sEH inhibitor.

FIG. 4. Number of neutrophils in BAL from rats exposed to tobacco smoke for 3 days. Rats were exposed to filtered air (grey bars) after treatment with vehicle, sEH inhibitor, or sEH inhibitor+EETs. Additional rats were exposed to tobacco smoke (black bars) after treatment with vehicle, sEH inhibitor, or sEH inhibitor+EETs. Data are presented as mean+SE (n=4). a p<0.05, compared to respective filtered air control. b p<0.05, compared to tobacco smoke+vehicle. c p<0.05, compared to tobacco smoke +sEH inhibitor.

FIG. 5. Number of lymphocytes in BAL from rats exposed to tobacco smoke for 3 days. Rats were exposed to filtered air (grey bars) after treatment with vehicle, sEH inhibitor, or sEH inhibitor+EETs. Additional rats were exposed to tobacco smoke (black bars) after treatment with vehicle, sEH inhibitor, or sEH inhibitor+EETs. Data are presented as mean+SE (n=4). a p<0.05, compared to respective filtered air control. b p<0.05, compared to tobacco smoke+vehicle. c p<0.05, compared to tobacco smoke+sEH inhibitor.

FIG. 6. Number of eosinophils in BAL from rats exposed to tobacco smoke for 3 days. Rats were exposed to filtered air (grey bars) after treatment with vehicle, sEH inhibitor, or sEH inhibitor+EETs. Additional rats were exposed to tobacco smoke (black bars) after treatment with vehicle, sEH inhibitor, or sEH inhibitor+EETs. Data are presented as mean+SE (n=4).

FIG. 7. FIG. 7 shows a process for the synthesis of EETs.

FIG. 8. FIG. 8 is a table showing the release rate of EETs from wax pellets.

### DETAILED DESCRIPTION

Introduction

As noted in the Background section, chronic obstructive pulmonary disease, or COPD, is a lung disease that is a leading cause of death in the United States and other countries. COPD encompasses two conditions, emphysema and chronic bronchitis, which relate to damage caused to the lung by air pollution, chronic exposure to chemicals, and tobacco smoke. Emphysema as a disease relates to damage to the alveoli of the lung, which results in loss of the separation between alveoli and a consequent reduction in the overall surface area available for gas exchange. Chronic bronchitis relates to irritation of the bronchioles, resulting in excess production of mucin, and the consequent blocking by mucin of the airways leading to the alveoli. While persons with emphysema do not necessarily have chronic bronchitis or vice versa, it is common for persons with one of the conditions to also have the other, as well as other lung disorders.

Surprisingly, it has now been discovered that some of the damage to the lungs due to COPD, emphysema, chronic bronchitis, and other obstructive lung disorders can be inhibited or reversed by administering inhibitors of the enzyme known as soluble epoxide hydrolase, or “sEH”. Even more surprisingly, it has now been discovered that the effects of sEH inhibitors can be increased by also administering cis-epoxyeicosantrienoic acids (“EETs”). The effect is at least additive over administering the two agents separately, and may indeed be synergistic. As discussed in more detail below, the results of the studies reported herein further indicate that the invention will be useful in reducing damage due to interstitial lung diseases and asthma.

EETs, which are epoxides of arachidonic acid, are known to be effectors of blood pressure, regulators of inflammation, and modulators of vascular permeability. Hydrolysis of the epoxides by sEH diminishes this activity. Inhibition of sEH raises the level of EETs since the rate at which the EETs are hydrolyzed into DHETs is reduced.

EETs useful in the methods of the present invention include 14,15-EET, 8,9-EET and 11,12-EET, and 5,6 EETs, in that order of preference. Preferably, the EETs are administered as the methyl ester, which is more stable. Persons of skill will recognize that the EETs are regioisomers, such as 8S,9R- and 14R,15S-EET. 8,9-EET, 11,12-EET, and 14R,15S-EET, are commercially available from, for example, Sigma-Aldrich (catalog nos. E5516, E5641, and E5766, respectively, Sigma-Aldrich Corp., St. Louis, Mo.).

EETs have not previously been administered therapeutically, largely because it has been believed they would be hydrolyzed too quickly by endogenous sEH to be helpful. It was not known whether endogenous sEH could be inhibited sufficiently in the lungs to permit administration of exogenous EET to result in increased levels of EETs over those normally present. In the studies underlying the present invention, administration of EETs in conjunction with inhibitors of sEH to rats exposed to tobacco smoke resulted in reduced levels of recruitment of white blood cells in the lungs than did administration of sEH inhibitors alone. The results indicate that the combination of the two agents was more powerful in reducing tobacco smoke-related irritation to the lung than administration of sEH inhibitor alone. (EETs were not administered by themselves since it was anticipated they would be degraded too quickly to have a useful effect.)

Thus, the studies reported herein show that EETs can be used in conjunction with sEH inhibitors to reduce damage to the lungs by tobacco smoke or, by extension, by occupational or environmental irritants. These findings indicate that the co-administration of sEH inhibitors and of EETs can be used to inhibit or slow the development or progression of COPD, emphysema, chronic bronchitis, or other chronic obstructive lung diseases which cause irritation to the lungs.

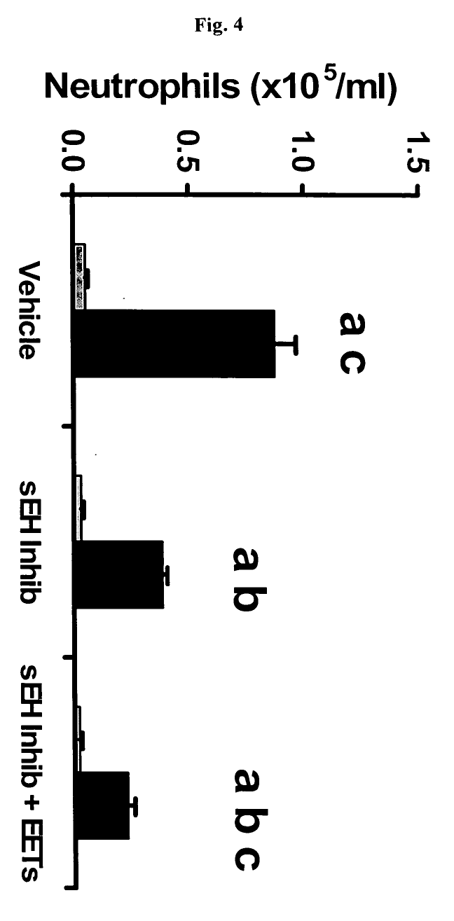

In our animal model of COPD and in humans, we have found that there are elevated levels of immunomodulatory lymphocytes and neutrophils. Neutrophils release agents that cause tissue damage and, if not regulated, will over time have a destructive effect. Without wishing to be bound by theory, it is believed that reducing levels of neutrophils reduces tissue damage contributing to obstructive lung diseases such as COPD, emphysema, and chronic bronchitis. In the studies reported in the Examples, the administration of sEH inhibitors to rats in an animal model of COPD resulted in approximately a 55% reduction in the number of neutrophils found in the lungs. Administration of EETs in addition to the sEH inhibitors reduced neutrophil levels by a total of some 73%. The reduction in neutrophil levels in the presence of sEH inhibitor and EETs was approximately 41% greater than in the presence of the sEH inhibitor alone. See, FIG. 4.

This is an important advance. While levels of endogenous EETs are expected to rise with the inhibition of sEH activity caused by the action of the sEH inhibitor, and therefore to result in at least some improvement in symptoms or pathology, it may not be sufficient in all cases to inhibit progression of COPD or other pulmonary diseases. This is particularly true where the diseases or other factors have reduced the endogenous concentrations of EETs below those normally present in healthy individuals. Administration of exogenous EETs in conjunction with an sEH inhibitor is therefore expected to augment the effects of the sEH inhibitor in inhibiting or reducing the progression of COPD or other pulmonary diseases.

In addition to inhibiting or reducing the progression of chronic obstructive airway conditions, the invention also provides new ways of reducing the severity or progression of chronic restrictive airway diseases. While obstructive airway diseases tend to result from the destruction of the lung parenchyma, and especially of the alveoli, restrictive diseases tend to arise from the deposition of excess collagen in the parenchyma. These restrictive diseases are commonly referred to as “interstitial lung diseases”, or “ILDs”, and include conditions such as idiopathic pulmonary fibrosis. The methods, compositions and uses of the invention are useful for reducing the severity or progression of ILDs, such as idiopathic pulmonary fibrosis. Macrophages play a significant role in stimulating interstitial cells, particularly fibroblasts, to lay down collagen. Without wishing to be bound by theory, it is believed that neutrophils are involved in activating macrophages, and that the reduction of neutrophil levels found in the studies reported herein demonstrate that the methods and uses of the invention will also be applicable to reducing the severity and progression of ILDs.

In some preferred embodiments, the ILD is idiopathic pulmonary fibrosis. In other preferred embodiments, the ILD is one associated with an occupational or environmental exposure. Exemplars of such ILDs, are asbestosis, silicosis, coal worker's pneumoconiosis, and berylliosis. Further, occupational exposure to any of a number of inorganic dusts and organic dusts is believed to be associated with mucus hypersecretion and respiratory disease, including cement dust, coke oven emissions, mica, rock dusts, cotton dust, and grain dust (for a more complete list of occupational dusts associated with these conditions, see Table 254-1 of Speizer, “Environmental Lung Diseases,” Harrison's Principles of Internal Medicine, infra, at pp. 1429-1436). In other embodiments, the ILD is sarcoidosis of the lungs. ILDs can also result from radiation in medical treatment, particularly for breast cancer, and from connective tissue or collagen diseases such as rheumatoid arthritis and systemic sclerosis. It is believed that the methods, uses and compositions of the invention can be useful in each of these interstitial lung diseases.

In another set of embodiments, the invention is used to reduce the severity or progression of asthma. Asthma typically results in mucin hypersecretion, resulting in partial airway obstruction. Additionally, irritation of the airway results in the release of mediators which result in airway obstruction. While the lymphocytes and other immunomodulatory cells recruited to the lungs in asthma may differ from those recruited as a result of COPD or an ILD, it is expected that the invention will reduce the influx of immunomodulatory cells, such as neutrophils and eosinophils, and ameliorate the extent of obstruction. Thus, it is expected that the administration of sEH inhibitors, and the administration of sEH inhibitors in combination with EETs, will be useful in reducing airway obstruction due to asthma.

In each of these diseases and conditions, it is believed that at least some of the damage to the lungs is due to agents released by neutrophils which infiltrate into the lungs. The presence of neutrophils in the airways is thus indicative of continuing damage from the disease or condition, while a reduction in the number of neutrophils is indicative of reduced damage or disease progression. Thus, a reduction in the number of neutrophils in the airways in the presence of an agent is a marker that the agent is reducing damage due to the disease or condition, and is slowing the further development of the disease or condition. The number of neutrophils present in the lungs can be determined by, for example, bronchoalveolar lavage.

Medicaments of EETs can be made which can be administered in conjunction with one or more sEH inhibitors, or a medicament containing one or more sEH inhibitors can optionally contain one or more EETs. The EETs can be administered concurrently with the sEH inhibitor, or following administration of the sEH inhibitor. It is understood that, like all drugs, inhibitors have half lives defined by the rate at which they are metabolized by or excreted from the body, and that the inhibitor will have a period following administration during which it will be present in amounts sufficient to be effective. If EETs are administered after the inhibitor is administered, therefore, it is desirable that the EETs be administered during the period during which the inhibitor will be present in amounts to be effective to delay hydrolysis of the EETs. Typically, the EET or EETs will be administered within 48 hours of administering an sEH inhibitor. Preferably, the EET or EETs are administered within 24 hours of the inhibitor, and even more preferably within 12 hours. In increasing order of desirability, the EET or EETs are administered within 10, 8, 6, 4, 2, hours, 1 hour, or one half hour after administration of the inhibitor. Most preferably, the EET or EETs are administered concurrently with the inhibitor.

In some embodiments, the sEH inhibitor may be a nucleic acid, such as a small interfering RNA (siRNA), which reduces expression of a gene encoding sEH. The EETs may be administered in combination with such a nucleic acid. Typically, a study will determine the time following administration of the nucleic acid before a decrease is seen in levels of sEH. The EET or EETs will typically then be administered a time calculated to be after the activity of the nucleic acid has resulted in a decrease in sEH levels.

In some embodiments, the EETs, the sEH inhibitor, or both, are provided in a material that permits them to be released over time to provide a longer duration of action. Slow release coatings are well known in the pharmaceutical art; the choice of the particular slow release coating is not critical to the practice of the present invention.

EETs are subject to degradation under acidic conditions. Thus, if the EETs are to be administered orally, it is desirable that they are protected from degradation in the stomach. Conveniently, EETs for oral administration may be coated to permit them to passage the acidic environment of the stomach into the basic environment of the intestines. Such coatings are well known in the art. For example, aspirin coated with so-called “enteric coatings” is widely available commercially. Such enteric coatings may be used to protect EETs during passage through the stomach. A exemplar coating is set forth in the Examples.

II. Definitions

Units, prefixes, and symbols are denoted in their Systeme International de Unites (SI) accepted form. Numeric ranges are inclusive of the numbers defining the range. Unless otherwise indicated, nucleic acids are written left to right in 5′ to 3′ orientation; amino acid sequences are written left to right in amino to carboxy orientation. The headings provided herein are not limitations of the various aspects or embodiments of the invention, which can be had by reference to the specification as a whole. Accordingly, the terms defined immediately below are more fully defined by reference to the specification in its entirety. Terms not defined herein have their ordinary meaning as understood by a person of skill in the art.

“cis-Epoxyeicosatrienoic acids” (“EETs”) are biomediators synthesized by cytochrome P450 epoxygenases.

“Epoxide hydrolases” (“EH;” EC 3.3.2.3) are enzymes in the alpha beta hydrolase fold family that add water to 3 membered cyclic ethers termed epoxides.

“Soluble epoxide hydrolase” (“sEH”) is an enzyme which in endothelial and smooth muscle cells converts EETs to dihydroxy derivatives called dihydroxyeicosatrienoic acids (“DHETs”). The cloning and sequence of the murine sEH is set forth in Grant et al., J. Biol. Chem. 268(23):17628-17633 (1993). The cloning, sequence, and accession numbers of the human sEH sequence are set forth in Beetham et al., Arch. Biochem. Biophys. 305(1):197-201 (1993). The amino acid sequence of human sEH is also set forth as SEQ ID NO:2 of U.S. Pat. No. 5,445,956; the nucleic acid sequence encoding the human sEH is set forth as nucleotides 42-1703 of SEQ ID NO: 1 of that patent. The evolution and nomenclature of the gene is discussed in Beetham et al., DNA Cell Biol. 14(1):61-71 (1995). Soluble epoxide hydrolase represents a single highly conserved gene product with over 90% homology between rodent and human (Arand et al., FEBS Lett., 338:251-256 (1994)). Unless otherwise specified, as used herein, the terms “soluble epoxide hydrolase” and “sEH” refer to human sEH.

Unless otherwise specified, as used herein, the term “sEH inhibitor” refers to an inhibitor of human sEH. Preferably, the inhibitor does not also inhibit the activity of microsomal epoxide hydrolase by more than 25% at concentrations at which the inhibitor inhibits sEH by at least 50%, and more preferably does not inhibit mEH by more than 10% at that concentration. For convenience of reference, unless otherwise required by context, the term “sEH inhibitor” as used herein encompasses prodrugs which are metabolized to active inhibitors of sEH. Further for convenience of reference, and except as otherwise required by context, reference herein to a compound as an inhibitor of sEH includes reference to derivatives of that compound (such as an ester of that compound) that retain activity as an sEH inhibitor.

By “physiological conditions” is meant an extracellular milieu having conditions (e.g., temperature, pH, and osmolarity) which allows for the sustenance or growth of a cell of interest.

Unless otherwise required by context, “administering” an EET and an sEH inhibitor to a person in need thereof includes administering an sEH inhibitor, followed by a later administration of an EET while an amount of sEH inhibitor is still present sufficient to reduce by at least 25% the rate of hydrolysis of the EET by sEH.

“Parenchyma” refers to the tissue characteristic of an organ, as distinguished from associated connective or supporting tissues.

“Chronic Obstructive Pulmonary Disease” or “COPD” is also sometimes known as “chronic obstructive airway disease”, “chronic obstructive lung disease”, and “chronic airways disease.” COPD is generally defined as a disorder characterized by reduced maximal expiratory flow and slow forced emptying of the lungs. COPD is considered to encompass two related conditions, emphysema and chronic bronchitis. COPD can be diagnosed by the general practitioner using art recognized techniques, such as the patient's forced vital capacity (“FVC”), the maximum volume of air that can be forceably expelled after a maximal inhalation. In the offices of general practitioners, the FVC is typically approximated by a 6 second maximal exhalation through a spirometer. The definition, diagnosis and treatment of COPD, emphysema, and chronic bronchitis are well known in the art and discussed in detail by, for example, Honig and Ingram, in Harrison's Principles of Internal Medicine, (Fauci et al., Eds.), 14th Ed., 1998, McGraw-Hill, New York, pp. 1451-1460 (hereafter, “Harrison's Principles of Internal Medicine”).

“Emphysema” is a disease of the lungs characterized by permanent destructive enlargement of the airspaces distal to the terminal bronchioles without obvious fibrosis.

“Chronic bronchitis” is a disease of the lungs characterized by chronic bronchial secretions which last for most days of a month, for three months a year, for two years.

As the names imply, “obstructive pulmonary disease” and “obstructive lung disease” refer to obstructive diseases, as opposed to restrictive diseases. These diseases particularly include COPD, bronchial asthma and small airway disease.

“Small airway disease.” There is a distinct minority of patients whose airflow obstruction is due, solely or predominantly to involvement of the small airways. These are defined as airways less than 2 mm in diameter and correspond to small cartilaginous bronchi, terminal bronchioles and respiratory bronchioles. Small airway disease (SAD) represents luminal obstruction by inflammatory and fibrotic changes that increase airway resistance. The obstruction may be transient or permanent.

The “interstitial lung diseases (ILDs)” are a group of conditions involving the alveolar walls, perialveolar tissues, and contiguous supporting structures. As discussed on the website of the American Lung Association, the tissue between the air sacs of the lung is the interstitium, and this is the tissue affected by fibrosis in the disease. Persons with the disease have difficulty breathing in because of the stiffness of the lung tissue but, in contrast to persons with obstructive lung disease, have no difficulty breathing out. The definition, diagnosis and treatment of interstitial lung diseases are well known in the art and discussed in detail by, for example, Reynolds, H. Y., in Harrison's Principles of Internal Medicine, supra, at pp. 1460-1466. Reynolds notes that, while ILDs have various initiating events, the immunopathological responses of lung tissue are limited and the ILDs therefore have common features.

“Idiopathic pulmonary fibrosis,” or “IPF,” is considered the prototype ILD. Although it is idiopathic in that the cause is not known, Reynolds, supra, notes that the term refers to a well defined clinical entity.

“Bronchoalveolar lavage,” or “BAL,” is a test which permits removal and examination of cells from the lower respiratory tract and is used in humans as a diagnostic procedure for pulmonary disorders such as IPF. In human patients, it is usually performed during bronchoscopy.

III. Inhibitors of Soluble Epoxide Hydrolase

Scores of sEH inhibitors are known, of a variety of chemical structures. Derivatives in which the urea, carbamate, or amide pharmacophore (as used herein, “pharmacophore” refers to the section of the structure of a ligand that binds to the sEH) is covalently bound to both an adamantane and to a 12 carbon chain dodecane are particularly useful as sEH inhibitors. Derivatives that are metabolically stable are preferred, as they are expected to have greater activity in vivo. Selective and competitive inhibition of sEH in vitro by a variety of urea, carbamate, and amide derivatives is taught, for example, by Morisseau et al., Proc. Natl. Acad. Sci. U. S. A, 96:8849-8854 (1999), which provides substantial guidance on designing urea derivatives that inhibit the enzyme.

Derivatives of urea are transition state mimetics that form a preferred group of sEH inhibitors. Within this group, N, N′-dodecyl-cyclohexyl urea (DCU), is preferred as an inhibitor, while N-cyclohexyl-N′-dodecylurea (CDU) is particularly preferred. Some compounds, such as dicyclohexylcarbodiimide (a lipophilic diimide), can decompose to an active urea inhibitor such as DCU. Any particular urea derivative or other compound can be easily tested for its ability to inhibit sEH by standard assays, such as those discussed herein. The production and testing of urea and carbamate derivatives as sEH inhibitors is set forth in detail in, for example, Morisseau et al., Proc Natl Acad Sci (USA) 96:8849-8854 (1999).

N-Adamantyl-N′-dodecyl urea (“ADU”) is both metabolically stable and has particularly high activity on sEH. (Both the 1- and the 2-admamantyl ureas have been tested and have about the same high activity as an inhibitor of sEH.) Thus, isomers of adamantyl dodecyl urea are particularly preferred inhibitors. It is further expected that other dodecanoic acid ester derivatives of urea are suitable for use in the methods of the invention.

U.S. Pat. No. 5,955,496 (the '496 patent) sets forth a number of suitable epoxide hydrolase inhibitors for use in the methods of the invention. One category of inhibitors comprises inhibitors that mimic the substrate for the enzyme. The lipid alkoxides (e.g., the 9-methoxide of stearic acid) are an exemplar of this group of inhibitors. In addition to the inhibitors discussed in the '496 patent, a dozen or more lipid alkoxides have been tested as sEH inhibitors, including the methyl, ethyl, and propyl alkoxides of oleic acid (also known as stearic acid alkoxides), linoleic acid, and arachidonic acid, and all have been found to act as inhibitors of sEH.

In another group of embodiments, the '496 patent sets forth sEH inhibitors that provide alternate substrates for the enzyme that are turned over slowly. Exemplars of this category of inhibitors are phenyl glycidols (e.g., S,S-4-nitrophenylglycidol), and chalcone oxides. The '496 patent notes that suitable chalcone oxides include 4-phenylchalcone oxide and 4-fluourochalcone oxide. The phenyl glycidols and chalcone oxides are believed to form stable acyl enzymes.

Additional inhibitors of sEH suitable for use in the methods of the invention are set forth in U.S. Pat. Nos. 6,150,415 (the '415 patent) and 6,531,506 (the '506 patent). Two preferred classes of inhibitors of the invention are compounds of Formulas 1 and 2, as described in the '415 and '506 patents. Means for preparing such compounds and assaying desired compounds for the ability to inhibit epoxide hydrolases are also described. The '506 patent, in particular, teaches scores of inhibitors of Formula 1 and some twenty inhibitors of Formula 2, which were shown to inhibit human sEH at concentrations as low as 0.1 μM. Any particular inhibitor can readily be tested to determine whether it will work in the methods of the invention by standard assays, such as that set forth in the Examples, below.

As noted above, chalcone oxides can serve as an alternate substrate for the enzyme. While chalcone oxides have half lives which depend in part on the particular structure, as a group the chalcone oxides tend to have relatively short half lives (a drug's half life is usually defined as the time for the concentration of the drug to drop to half its original value. See, e.g., Thomas, G., Medicinal Chemistry: an introduction, John Wiley & Sons Ltd. (West Sussex, England, 2000)). Since the uses of the invention contemplate inhibition of sEH over periods of time which can be measured in days, weeks, or months, chalcone oxides, and other inhibitors which have a half life whose duration is shorter than the practitioner deems desirable, are preferably administered in a manner which provides the agent over a period of time. For example, the inhibitor can be provided in materials that release the inhibitor slowly, including materials that release the inhibitor in or near the kidney, to provide a high local concentration. Methods of administration that permit high local concentrations of an inhibitor over a period of time are known, and are not limited to use with inhibitors which have short half lives although, for inhibitors with a relatively short half life, they are a preferred method of administration.

In addition to the compounds in Formula 1 of the '506 patent, which interact with the enzyme in a reversible fashion based on the inhibitor mimicking an enzyme-substrate transition state or reaction intermediate, one can have compounds that are irreversible inhibitors of the enzyme. The active structures such as those in the Tables or Formula 1 of the '506 patent can direct the inhibitor to the enzyme where a reactive functionality in the enzyme catalytic site can form a covalent bond with the inhibitor. One group of molecules which could interact like this would have a leaving group such as a halogen or tosylate which could be attacked in an SN2 manner with a lysine or histidine. Alternatively, the reactive functionality could be an epoxide or Michael acceptor such as an α/β-unsaturated ester, aldehyde, ketone, ester, or nitrile.

Further, in addition to the Formula 1 compounds, active derivatives can be designed for practicing the invention. For example, dicyclohexyl thio urea can be oxidized to dicyclohexylcarbodiimide which, with enzyme or aqueous acid (physiological saline), will form an active dicyclohexylurea. Alternatively, the acidic protons on carbamates or ureas can be replaced with a variety of substituents which, upon oxidation, hydrolysis or attack by a nucleophile such as glutathione, will yield the corresponding parent structure. These materials are known as prodrugs or protoxins (Gilman et al., The Pharmacological Basis of Therapeutics, 7th Edition, MacMillan Publishing Company, New York, p. 16 (1985)) Esters, for example, are common prodrugs which are released to give the corresponding alcohols and acids enzymatically (Yoshigae et al., Chirality, 9:661-666 (1997)). The drugs and prodrugs can be chiral for greater specificity. These derivatives have been extensively used in medicinal and agricultural chemistry to alter the pharmacological properties of the compounds such as enhancing water solubility, improving formulation chemistry, altering tissue targeting, altering volume of distribution, and altering penetration. They also have been used to alter toxicology profiles.

There are many prodrugs possible, but replacement of one or both of the two active hydrogens in the ureas described here or the single active hydrogen present in carbamates is particularly attractive. Such derivatives have been extensively described by Fukuto and associates. These derivatives have been extensively described and are commonly used in agricultural and medicinal chemistry to alter the pharmacological properties of the compounds. (Black et al., Journal of Agricultural and Food Chemistry, 21(5):747-751 (1973); Fahmy et al, Journal of Agricultural and Food Chemistry, 26(3):550-556 (1978); Jojima et al., Journal of Agricultural and Food Chemistry, 31(3):613-620 (1983); and Fahmy et al., Journal of Agricultural and Food Chemistry, 29(3):567-572 (1981).)

Such active proinhibitor derivatives are within the scope of the present invention, and the just-cited references are incorporated herein by reference. Without being bound by theory, it is believed that suitable inhibitors of the invention mimic the enzyme transition state so that there is a stable interaction with the enzyme catalytic site. The inhibitors appear to form hydrogen bonds with the nucleophilic carboxylic acid and a polarizing tyrosine of the catalytic site.

In some embodiments, sEH inhibition can include the reduction of the amount of sEH. As used herein, therefore, sEH inhibitors can therefore encompass nucleic acids that inhibit expression of a gene encoding sEH. Many methods of reducing the expression of genes, such as reduction of transcription and siRNA, are known, and are discussed in more detail below.

Preferably, the inhibitor inhibits sEH without also significantly inhibiting microsomal epoxide hydrolase (“mEH”). Preferably, at concentrations of 500 μM, the inhibitor inhibits sEH activity by at least 50% while not inhibiting mEH activity by more than 10%. Preferred compounds have an IC50 (inhibition potency or, by definition, the concentration of inhibitor which reduces enzyme activity by 50%) of less than about 500 μM. Inhibitors with IC50s of less than 500 μM are preferred, with IC50s of less than 100 μM being more preferred and IC50s of 50 μM, 40 μM, 30 μM, 25 μM, 20 μM, 15 μM, 10 μM, 5 μM, 3 μM, 2 μM, 1 μM or even less being the more preferred as the IC50 decreases. Assays for determining EH activity are known in the art and described elsewhere herein.

IV. EETs

EETs can be administered to inhibit the development or worsening of COPD. In preferred embodiments, one or more EETs are administered concurrently or after administration of an sEH inhibitor so that the EET or EETs are not hydrolyzed quickly.

Optionally, the EET or EETs are embedded or otherwise placed in a material that releases the EET over time. Materials suitable for promoting the slow release of compositions such as EETs are known in the art.

Conveniently, the EET or EETs can be administered orally. Since EETs are subject to degradation under acidic conditions, EETs intended for oral administration can be coated with a coating resistant to dissolving under acidic conditions, but which dissolve under the mildly basic conditions present in the intestines. Suitable coatings, commonly known as “enteric coatings” are widely used for products, such as aspirin, which cause gastric distress or which would undergo degradation upon exposure to gastric acid. By using coatings with an appropriate dissolution profile, the coated substance can be released in a chosen section of the intestinal tract. For example, a substance to be released in the colon is coated with a substance that dissolves at pH 6.5-7, while substances to be released in the duodenum can be coated with a coating that dissolves at pH values over 5.5. Such coatings are commercially available from, for example, Rohm Specialty Acrylics (Rohm America LLC, Piscataway, NJ) under the trade name “Eudragit®”. The choice of the particular enteric coating is not critical to the practice of the invention.

Preferred EETs include 14,15-EET, 8,9-EET and 11,12-EET in that order of preference. Purified sEH selected 8S,9R- and 14R,15S-EET; accordingly these EETs are particularly preferred. 8,9-EET, 11,12-EET, and 14R,15S-EET are commercially available from, for example, Sigma-Aldrich (catalog nos. E5516, E5641, and E5766, respectively, Sigma-Aldrich Corp., St. Louis, Mo.).

V. Assays for Epoxide Hydrolase Activity

Any of a number of standard assays for determining epoxide hydrolase activity can be used to determine inhibition of sEH. For example, suitable assays are described in Gill,. et al., Anal Biochem 131, 273-282 (1983); and Borhan, et al., Analytical Biochemistry 231, 188-200 (1995)). Suitable in vitro assays are described in Zeldin et al., J Biol. Chem. 268:6402-6407 (1993). Suitable in vivo assays are described in Zeldin et al., Arch Biochem Biophys 330:87-96 (1996). Assays for epoxide hydrolase using both putative natural substrates and surrogate substrates have been reviewed (see, Hammock, et al. In: Methods in Enzymology, Volume III, Steroids and Isoprenoids, Part B, (Law, J. H. and H. C. Rilling, eds. 1985), Academic Press, Orlando, Florida, pp. 303-311 and Wixtrom et al., In: Biochemical Pharmacology and Toxicology, Vol. 1: Methodological Aspects of Drug Metabolizing Enzymes, (Zakim, D. and D.A. Vessey, eds. 1985), John Wiley & Sons, Inc., New York, pp. 1-93. Several spectral based assays exist based on the reactivity or tendency of the resulting diol product to hydrogen bond (see, e.g., Wixtrom, supra, and Hammock. Anal. Biochem. 174:291-299 (1985) and Dietze, et al. Anal. Biochem. 216:176-187 (1994)).

The enzyme also can be detected based on the binding of specific ligands to the catalytic site which either immobilize the enzyme or label it with a probe such as dansyl, fluoracein, luciferase, green fluorescent protein or other reagent. The enzyme can be assayed by its hydration of EETs, its hydrolysis of an epoxide to give a colored product as described by Dietze et al., 1994, supra, or its hydrolysis of a radioactive surrogate substrate (Borhan et al., 1995, supra). The enzyme also can be detected based on the generation of fluorescent products following the hydrolysis of the epoxide. Numerous method of epoxide hydrolase detection have been described (see, e.g., Wixtrom, supra).

The assays are normally carried out with a recombinant enzyme following affinity purification. They can be carried out in crude tissue homogenates, cell culture or even in vivo, as known in the art and described in the references cited above..

VI. Other Means of inhibiting sEH activity

Other means of inhibiting sEH activity or gene expression can also be used in the methods of the invention. For example, a nucleic acid molecule complementary to at least a portion of the human sEH gene can be used to inhibit sEH gene expression. Means for inhibiting gene expression using, for example, short interfering RNA (siRNA), are known. “RNA interference”, a form of post-transcriptional gene silencing (“PTGS”), describes effects that result from the introduction of double-stranded RNA into cells (reviewed in Fire, A. Trends Genet 15:358-363 (1999); Sharp, P. Genes Dev 13:139-141 (1999); Hunter, C. Curr Biol 9:R440-R442 (1999); Baulcombe. D. Curr Biol 9:R599-R601 (1999); Vaucheret et al. Plant J 16: 651-659 (1998)). RNA interference, commonly referred to as RNAi, offers a way of specifically inactivating a cloned gene, and is a powerful tool for investigating gene function.

The active agent in RNAi is a long double-stranded (antiparallel duplex) RNA, with one of the strands corresponding or complementary to the RNA which is to be inhibited. The inhibited RNA is the target RNA. The long double stranded RNA is chopped into smaller duplexes of approximately 20 to 25 nucleotide pairs, after which the mechanism by which the smaller RNAs inhibit expression of the target is largely unknown at this time. While RNAi was shown initially to work well in lower eukaryotes, for mammalian cells, it was thought that RNAi might be suitable only for studies on the oocyte and the preimplantation embryo. In mammalian cells other than these, however, longer RNA duplexes provoked a response known as “sequence non-specific RNA interference,” characterized by the non-specific inhibition of protein synthesis.

Further studies showed this effect to be induced by dsRNA of greater than about 30 base pairs, apparently due to an interferon response. It is thought that dsRNA of greater than about 30 base pairs binds and activates the protein PKR and 2′,5′-oligonucleotide synthetase (2′,5′-AS). Activated PKR stalls translation by phosphorylation of the translation initiation factors eIF2α, and activated 2′,5′-AS causes mRNA degradation by 2′,5′-oligonucleotide-activated ribonuclease L. These responses are intrinsically sequence-nonspecific to the inducing dsRNA; they also frequently result in apoptosis, or cell death. Thus, most somatic mammalian cells undergo apoptosis when exposed to the concentrations of dsRNA that induce RNAi in lower eukaryotic cells.

More recently, it was shown that RNAi would work in human cells if the RNA strands were provided as pre-sized duplexes of about 19 nucleotide pairs, and RNAi worked particularly well with small unpaired 3′ extensions on the end of each strand (Elbashir et al. Nature 411: 494-498 (2001)). In this report, “short interfering RNA” (siRNA, also referred to as small interfering RNA) were applied to cultured cells by transfection in oligofectamine micelles. These RNA duplexes were too short to elicit sequence-nonspecific responses like apoptosis, yet they efficiently initiated RNAI. Many laboratories then tested the use of siRNA to knock out target genes in mammalian cells. The results demonstrated that siRNA works quite well in most instances.

For purposes of reducing the activity of sEH, siRNAs to the gene encoding sEH can be specifically designed using computer programs. The cloning, sequence, and accession numbers of the human sEH sequence are set forth in Beetham et al., Arch. Biochem. Biophys. 305(1):197-201 (1993). The amino acid sequence of human sEH is also set forth as SEQ ID NO:2 of U.S. Pat. No. 5,445,956; nucleotides 42-1703 of SEQ ID NO:1 are the nucleic acid sequence encoding the amino acid sequence.

A program, siDESIGN from Dharmacon, Inc. (Lafayette, CO), permits predicting siRNAs for any nucleic acid sequence, and is available on the World Wide Web at dharmacon.com. Programs for designing siRNAs are also available from others, including Genscript (available on the Web at genscript.com/ssl-bin/app/rnai) and, to academic and non-profit researchers, from the Whitehead Institute for Biomedical Research on the internet by entering “http://” followed by “jura.wi.mit.edu/pubint/http://iona.wi.mit.edu/siRNAext/.”

For example, using the program available from the Whitehead Institute, the following sEH target sequences and siRNA sequences can be generated:

| | |
|---|---|
| 1) Target: | |
| CAGTGTTCATTGGCCATGACTGG | (SEQ ID NO:3) |
| Sense-siRNA: | |
| 5′ - GUGUUCAUUGGCCAUGACUTT- 3′ | (SEQ ID NO:4) |
| Antisense-siRNA: | |
| 5′ - AGUCAUGGCCAAUGAACACTT- 3′ | (SEQ ID NO:5) |
| 2) Target: | |
| GAAAGGCTATGGAGAGTCATCTG | (SEQ ID NO:6) |
| Sense-siRNA: | |
| 5′ - AAGGCUAUGGAGAGUCAUCTT- 3′ | (SEQ ID NO:7) |
| Antisense-siRNA: | |
| 5′ - GAUGACUCUCCAUAGCCUUTT- 3′ | (SEQ ID NO:8) |
| 3) Target | |
| AAAGGCTATGGAGAGTCATCTGC | (SEQ ID NO:9) |
| Sense-siRNA: | |
| 5′ - AGGCUAUGGAGAGUCAUCUTT- 3′ | (SEQ ID NO:10) |
| Antisense-siRNA: | |
| 5′ - AGAUGACUCUCCAUAGCCUTT- 3′ | (SEQ ID NO:11) |
| 4) Target: | |
| CAAGCAGTGTTCATTGGCCATGA | (SEQ ID NO:12) |
| Sense-siRNA: | |
| 5′ - AGCAGUGUUCAUUGGCCAUTT- 3′ | (SEQ ID NO:13) |
| Antisense-siRNA: | |
| 5′ - AUGGCCAAUGAACACUGCUTT- 3′ | (SEQ ID NO:14) |
| 5) Target: | |
| CAGCACATGGAGGACTGGATTCC | (SEQ ID NO:15) |
| Sense-siRNA: | |
| 5′ - GCACAUGGAGGACUGGAUUTT- 3′ | (SEQ ID NO:16) |
| Antisense-siRNA: | |
| 5′ - AAUCCAGUCCUCCAUGUGCTT- 3′ | (SEQ ID NO:17) |

Alternatively, siRNA can be generated using kits which generate siRNA from the gene. For example, the “Dicer siRNA Generation” kit (catalog number T510001, Gene Therapy Systems, Inc., San Diego, Calif.) uses the recombinant human enzyme “dicer” in vitro to cleave long double stranded RNA into 22 bp siRNAs. By having a mixture of siRNAs, the kit permits a high degree of success in generating siRNAs that will reduce expression of the target gene. Similarly, the Silencer™ siRNA Cocktail Kit (RNase III) (catalog no. 1625, Ambion, Inc., Austin, Tex.) generates a mixture of siRNAs from dsRNA using RNase III instead of dicer. Like dicer, RNase III cleaves dsRNA into 12-30 bp dsRNA fragments with 2 to 3 nucleotide 3′overhangs, and 5′-phosphate and 3′-hydroxyl termini. According to the manufacturer, dsRNA is produced using T7 RNA polymerase, and reaction and purification components included in the kit. The dsRNA is then digested by RNase III to create a population of siRNAs. The kit includes reagents to synthesize long dsRNAs by in vitro transcription and to digest those dsRNAs into siRNA-like molecules using RNase III. The manufacturer indicates that the user need only supply a DNA template with opposing T7 phage polymerase promoters or two separate templates with promoters on opposite ends of the region to be transcribed.

The siRNAs can also be expressed from vectors. Typically, such vectors are administered in conjunction with a second vector encoding the corresponding complementary strand. Once expressed, the two strands anneal to each other and form the functional double stranded siRNA. One exemplar vector suitable for use in the invention is pSuper, available from OligoEngine, Inc. (Seattle, WA). In some embodiments, the vector contains two promoters, one positioned downstream of the first and in antiparallel orientation. The first promoter is transcribed in one direction, and the second in the direction antiparallel to the first, resulting in expression of the complementary strands. In yet another set of embodiments, the promoter is followed by a first segment encoding the first strand, and a second segment encoding the second strand. The second strand is complementary to the palindrome of the first strand. Between the first and the second strands is a section of RNA serving as a linker (sometimes called a “spacer”) to permit the second strand to bend around and anneal to the first strand, in a configuration known as a “hairpin.”

The formation of hairpin RNAs, including use of linker sections, is well known in the art. Typically, an siRNA expression cassette is employed, using a Polymerase III promoter such as human U6, mouse U6, or human HI. The coding sequence is typically a 19-nucleotide sense siRNA sequence linked to its reverse complementary antisense siRNA sequence by a short spacer. Nine-nucleotide spacers are typical, although other spacers can be designed. For example, the Ambion website indicates that its scientists have had success with the spacer TTCAAGAGA (SEQ ID NO: 18). Further, 5-6 T's are often added to the 3′ end of the oligonucleotide to serve as a termination site for Polymerase III. See also, Yu et al., Mol Ther 7(2):228-36 (2003); Matsukura et al., Nucleic Acids Res 31(15):e77 (2003).

As an example, the siRNA targets identified above can be targeted by hairpin siRNA as follows. And if you would like to attack the same targets by short hairpin RNAs, produced by a vector (permanent RNAi effect) you would put sense and antisense strand in a row with a loop forming sequence in between and suitable sequences for an adequate expression vector to both ends of the sequence. The ends of course depend on the cutting sites of the vector. The following are non-limiting examples of hairpin sequences that can be cloned into the pSuper vector:

(SEQ ID NO:19)

1) Target: CAGTGTTCATTGGCCATGACTGG

(SEQ ID NO:20)

Sense strand: 5′-
GATCCCCGTGTTCATTGGCCATGACTTTCAAGAGAAGTCATGGCCAATGA
ACACTTTTT-3′

(SEQ ID NO:21)

Antisense strand: 5′-
AGCTAAAAAGTGTTCATTGGCCATGACTTCTCTTGAAAGTCATGGCCAAT
GAACACGGG -3′

(SEQ ID NO:22)

2) Target: GAAAGGCTATGGAGAGTCATCTG

(SEQ ID NO:23)

Sense strand: 5′-
GATCCCCAAGGCTATGGAGAGTCATCTTCAAGAGAGATGACTCTCCATAG
CCTTTTTTT -3′

(SEQ ID NO:24)

Antisense strand: 5′-
AGCTAAAAAAAGGCTATGGAGAGTCATCTCTCTTGAAGATGACTCTCCAT
AGCCTTGGG -3′

(SEQ ID NO:25)

3) Target: AAAGGCTATGGAGAGTCATCTGC

(SEQ ID NO:26)

Sense strand: 5′-
GATCCCCAGGCTATGGAGAGTCATCTTTCAAGAGAAGATGACTCTCCATA
GCCTTTTTT -3′

(SEQ ID NO:27)

Antisense strand: 5′-
AGCTAAAAAAGGCTATGGAGAGTCATCATCTCTTGAAAGATGACTCTCCA
TAGCCTGGG -3′

(SEQ ID NO:28)

4) Target: CAAGCAGTGTTCATTGGCCATGA

(SEQ ID NO:29)

Sense strand: 5′-
GATCCCCAGCAGTGTTCATTGGCCATTTCAAGAGAATGGCCAATGAACAC
TGCTTTTTT -3′

(SEQ ID NO:30)

Antisense strand: 5′-
AGCTAAAAAAGCAGTGTTCATTGGCCATTCTCTTGAAATGGCCAATGAAC
ACTGCTGGG -3′

(SEQ ID NO:31)

5) Target: CAGCACATGGAGGACTGGATTCC

(SEQ ID NO:32)

Sense strand 5′-
GATCCCCGCACATGGAGGACTGGATTTTCAAGAGAAATCCAGTCCTCCAT
GTGCTTTTT -3′

(SEQ ID NO:33)

Antisense strand: 5′-
AGCTAAAAAGCACATGGAGGACTGGATTTCTCTTGAAAATCCAGTCCTCC
ATGTGCGGG -3′

In addition to siRNAs, other means are known in the art for inhibiting the expression of antisense molecules, ribozymes, and the like are well known to those of skill in the art. The nucleic acid molecule can be a DNA probe, a riboprobe, a peptide nucleic acid probe, a phosphorothioate probe, or a 2′-O methyl probe.

Generally, to assure specific hybridization, the antisense sequence is substantially complementary to the target sequence. In certain embodiments, the antisense sequence is exactly complementary to the target sequence. The antisense polynucleotides may also include, however, nucleotide substitutions, additions, deletions, transitions, transpositions, or modifications, or other nucleic acid sequences or non-nucleic acid moieties so long as specific binding to the relevant target sequence corresponding to the sEH gene is retained as a functional property of the polynucleotide. In one embodiment, the antisense molecules form a triple helix-containing, or “triplex” nucleic acid. Triple helix formation results in inhibition of gene expression by, for example, preventing transcription of the target gene (see, e.g., Cheng et al., 1988, J. Biol. Chem. 263:15110; Ferrin and Camerini-Otero, 1991, Science 354:1494; Ramdas et al., 1989, J. Biol. Chem. 264:17395; Strobel et al., 1991, Science 254:1639; and Rigas et al., 1986, Proc. Natl. Acad. Sci. U.S.A. 83:9591)

Antisense molecules can be designed by methods known in the art. For example, Integrated DNA Technologies (Coralville, Iowa) makes available a program on the internet which can be found by entering http://, followed by biotools.idtdna.com/antisense/AntiSense.aspx, which will provide appropriate antisense sequences for nucleic acid sequences up to 10,000 nucleotides in length. Using this program with the sEH gene provides the following exemplar sequences:

| | |
|---|---|
| 1) UGUCCAGUGCCCACAGUCCU | (SEQ ID NO:34) |
| 2) UUCCCACCUGACACGACUCU | (SEQ ID NO:35) |
| 3) GUUCAGCCUCAGCCACUCCU | (SEQ ID NO:36) |
| 4) AGUCCUCCCGCUUCACAGA | (SEQ ID NO:37) |
| 5) GCCCACUUCCAGUUCCUUUCC | (SEQ ID NO:38) |

In another embodiment, ribozymes can be designed to cleave the mRNA at a desired position. (See, e.g., Cech, 1995, Biotechnology 13:323; and Edgington, 1992, Biotechnology 10:256 and Hu et al., PCT Publication WO 94/03596).

The antisense nucleic acids (DNA, RNA, modified, analogues, and the like) can be made using any suitable method for producing a nucleic acid, such as the chemical synthesis and recombinant methods disclosed herein and known to one of skill in the art. In one embodiment, for example, antisense RNA molecules of the invention may be prepared by de novo chemical synthesis or by cloning. For example, an antisense RNA can be made by inserting (ligating) a sEH gene sequence in reverse orientation operably linked to a promoter in a vector (e.g., plasmid). Provided that the promoter and, preferably termination and polyadenylation signals, are properly positioned, the strand of the inserted sequence corresponding to the noncoding strand will be transcribed and act as an antisense oligonucleotide of the invention.

It will be appreciated that the oligonucleotides can be made using nonstandard bases (e.g., other than adenine, cytidine, guanine, thymine, and uridine) or nonstandard backbone structures to provides desirable properties (e.g., increased nuclease-resistance, tighter-binding, stability or a desired Tm). Techniques for rendering oligonucleotides nuclease-resistant include those described in PCT Publication WO 94/12633. A wide variety of useful modified oligonucleotides may be produced, including oligonucleotides having a peptide-nucleic acid (PNA) backbone (Nielsen et al., 1991, Science 254:1497) or incorporating 2′-O methyl ribonucleotides, phosphorothioate nucleotides, methyl phosphonate nucleotides, phosphotriester nucleotides, phosphorothioate nucleotides, phosphoramidates.

Proteins have been described that have the ability to translocate desired nucleic acids across a cell membrane. Typically, such proteins have amphiphilic or hydrophobic subsequences that have the ability to act as membrane-translocating carriers. For example, homeodomain proteins have the ability to translocate across cell membranes. The shortest intemalizable peptide of a homeodomain protein, Antennapedia, was found to be the third helix of the protein, from amino acid position 43 to 58 (see, e.g., Prochiantz, 1996, Current Opinion in Neurobiology 6:629-634. Another subsequence, the h (hydrophobic) domain of signal peptides, was found to have similar cell membrane translocation characteristics (see, e.g., Lin et al., 1995, J. Biol. Chem. 270:14255-14258). Such subsequences can be used to translocate oligonucleotides across a cell membrane. Oligonucleotides can be conveniently derivatized with such sequences. For example, a linker can be used to link the oligonucleotides and the translocation sequence. Any suitable linker can be used, e.g., a peptide linker or any other suitable chemical linker.

VII. Therapeutic Administration

EETs and inhibitors of sEH can be prepared and administered in a wide variety of ora 1, parenteral and topical dosage forms. In preferred forms, compounds for use in the methods of the present invention can be administered by injection, that is, intravenously, intramuscularly, intracutaneously, subcutaneously, intraduodenally, or intraperitoneally. The sEH inhibitor or EETs, or both, can also be administered by inhalation, for example, intranasally. Additionally, the sEH inhibitors, or EETs, or both, can be administered transdermally. Accordingly, the methods of the invention permit administration of pharmaceutical compositions comprising a pharmaceutically acceptable carrier or excipient and either a selected inhibitor or a pharmaceutically acceptable salt of the inhibitor.

For preparing pharmaceutical compositions from sEH inhibitors, or EETs, or both, pharmaceutically acceptable carriers can be either solid or liquid. Solid form preparations include powders, tablets, pills, capsules, cachets, suppositories, and dispersible granules. A solid carrier can be one or more substances which may also act as diluents, flavoring agents, binders, preservatives, tablet disintegrating agents, or an encapsulating material.

In powders, the carrier is a finely divided solid which is in a mixture with the finely divided active component. In tablets, the active component is mixed with the carrier having the necessary binding properties in suitable proportions and compacted in the shape and size desired. The powders and tablets preferably contain from 5% or 10% to 70% of the active compound. Suitable carriers are magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. The term “preparation” is intended to include the formulation of the active compound with encapsulating material as a carrier providing a capsule in which the active component with or without other carriers, is surrounded by a carrier, which is thus in association with it. Similarly, cachets and lozenges are included. Tablets, powders, capsules, pills, cachets, and lozenges can be used as solid dosage forms suitable for oral administration.

For preparing suppositories, a low melting wax, such as a mixture of fatty acid glycerides or cocoa butter, is first melted and the active component is dispersed homogeneously therein, as by stirring. The molten homogeneous mixture is then poured into convenient sized molds, allowed to cool, and thereby to solidify.

Liquid form preparations include solutions, suspensions, and emulsions, for example, water or water/propylene glycol solutions. For parenteral injection, liquid preparations can be formulated in solution in aqueous polyethylene glycol solution. Transdermal administration can be performed using suitable carriers. If desired, apparatuses designed to facilitate transdermal delivery can be employed. Suitable carriers and apparatuses are well known in the art, as exemplified by U.S. Pat. Nos. 6,635,274, 6,623,457, 6,562,004, and 6,274,166.

Aqueous solutions suitable for oral use can be prepared by dissolving the active component in water and adding suitable colorants, flavors, stabilizers, and thickening agents as desired. Aqueous suspensions suitable for oral use can be made by dispersing the finely divided active component in water with viscous material, such as natural or synthetic gums, resins, methylcellulose, sodium carboxymethylcellulose, and other well-known suspending agents.

Also included are solid form preparations which are intended to be converted, shortly before use, to liquid form preparations for oral administration. Such liquid forms include solutions, suspensions, and emulsions. These preparations may contain, in addition to the active component, colorants, flavors, stabilizers, buffers, artificial and natural sweeteners, dispersants, thickeners, solubilizing agents, and the like.

The pharmaceutical preparation is preferably in unit dosage form. In such form the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form.

The term “unit dosage form”, as used in the specification, refers to physically discrete units suitable as unitary dosages for human subjects and animals, each unit containing a predetermined quantity of active material calculated to produce the desired pharmaceutical effect in association with the required pharmaceutical diluent, carrier or vehicle. The specifications for the novel unit dosage forms of this invention are dictated by and directly dependent on (a) the unique characteristics of the active material and the particular effect to be achieved and (b) the limitations inherent in the art of compounding such an active material for use in humans and animals, as disclosed in detail in this specification, these being features of the present invention.

A therapeutically effective amount of the sEH inhibitor, or EETs, or both,is employed in slowing or inhibiting lung inflammation, COPD, or both. The dosage of the specific compound for treatment depends on many factors that are well known to those skilled in the art. They include for example, the route of administration and the potency of the particular compound. An exemplary dose is from about 0.001 μM/kg to about 100 mg/kg body weight of the mammal.

EETs are unstable, and can be converted to DHET in acidic conditions, such as those in the stomach. To avoid this, EETs can be administered intravenously or by injection. EETs intended for oral administration can be encapsulated in a coating that protects the EETs during passage through the stomach. For example, the EETs can be provided with a so-called “enteric” coating, such as those used for some brands of aspirin, or embedded in a formulation. Such enteric coatings and formulations are well known in the art. In some formulations, the EETs, or a combination of the EETs and an sEH inhibitor are embedded in a slow-release formulation to facilitate administration of the agents over time.

Without further elaboration, it is believed that one skilled in the art can, using the preceding description, practice the present invention to its fullest extent.

### EXAMPLES

The following examples are offered to illustrate, but not to limit the claimed invention.

### Example 1

Materials and Methods

Reagents and Chemicals.

12-(3-adamantane-1-yl-ureido)-dodecanoic acid butyl ester (AUDA-nBE) and 1-cyclohexyl-3-tetradecyl urea (CTU, Internal standard) were synthesized in our laboratory. These products were purified by recrystallization and characterized structurally by ¹H- and/or ¹³C-NMR, infrared, and mass spectroscopy. HPLC-grade methanol, acetonitrile and ethyl acetate were purchased from EMD Chemicals Inc. (Gibbstown, N.J.). Formic acid was obtained from Sigma-Aldrich (St. Louis, Mo.). Water (>18.0 MΩ) used was purified by NANO pure II system (Barnstead, Newton, Mass.).

Equipment.

LC-MS-MS analysis was performed using a Micromass Quattro Ultima triple quadrupole tandem mass spectrometer (Micromass, Manchester, UK) equipped with atomospheric pressure ionization source [atomospheric z-spray pressure chemical ionization (APcl) or electrospray ionization (ESI) interface]. The HPLC system consisted of a Waters model 2790 separations module (Waters Corporation, Milford, Mass.) including an autosampler with refrigerated sample compartment and as inline vacuum degasser and Waters model 2487 dual λ absorbance detector (Waters Corporation). An XTerra™MS C18 column (30×2.1 mm I. D., 3.5 μm; Waters Corporation) was used with a flow rate of 0.3 mL/min at ambient temperature. Data were manipulated with MassLynx software (Ver. 4.0).

LC-MS-MS conditions.

The ESI mass spectrometer was operated in the positive ion mode with a capillary voltage at 1.0 kV. Cone gas (N₂) and desolvation gas (N₂) were maintained at flow rates of 130 and 630 L/h, respectively. The source and the desolvation temperature were set at 100° C. and 300° C., respectively. The optimum cone voltages were set at 50 V for AUDA-NBE, 80 V for AUDA and 100 V for CTU (internal standard), respectively. Quantitative analysis was performed in the multiple reaction monitoring (MRM) mode with a dwell time of 300 ms. Ultra pure argon (99.9999%) was used as a collision gas at a pressure of 2.5 milli-torr for collision-induced dissociation (CID). Chromatographic separation was performed using a two-solvent linear gradient system. Solvents A and B used were 0.1% formic acid and acetonitrile containing 0.1% formic acid, respectively. Solvents were filtered through 0.45 μm membrane and degassed before use. Mobile phases were mixed with a linear gradient from 40% B to 100% B over 0-5 min, and then isocratic for 8 min with 100% B. The postrun was carried out to equilibrate the column to the initial conditions for 1 min before next run. Ten microliters of standard and the extracted blood samples were injected onto the column.

Making EETs wax plug.

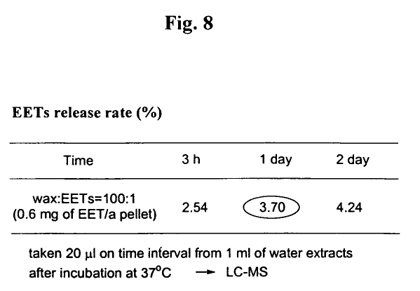

To create a wax pellet, the wax was melted at 100° C. for 20 min using a hot plate and the EETs were added to the molten wax while stirring. The wax-EETs suspension was then poured into a mold made with glass plates and then cooled to room temperature. The resultant wax stick containing EETs was cut to suitable size. To investigate the release rate of EETs from the resulting wax pellets in vitro, pellets (60 mg pellet containing 600 μg of EETs) were incubated at 37° C. in purified water (1 ml) containing an antioxidant. Aliquots (20 μl ) were taken at various time intervals. Each aliquot was added to 30 μl of MeOH containing an internal standard and injected into LC-MS to determine the concentration of EETs in the aliquot. The results are shown as a table in FIG. 8.

Enteric coating of EETs for oral administration.

Enteric dosage form is one of the most useful methods for the delivery of acid sensitive drugs. To investigate the biological effects of EETs in separating with that of DHET, we also developed oral administration with enteric coated EETs particles to eliminate dissolution in the stomach.

The particles consisted of lactose, EETs and an enteric coating polymer of cellulose acetate phthalate in the ratio of 2.0:0.1:0.4. To the lactose powder used as a core, EETs were added dropwise with mixing and then an acetone or EtOAC/EtOH solution of enteric coating polymer was added dropwise to this mixture. Drying in vacco gave the enteric coated EETs particles with a range of 200-360 nm as a suitable size powder for oral administration of mice and rats.

Dissolution tests were performed in water, acidic and pH 7.4 buffer solution. Ten mg of each particle were added to 1 ml of 0.1 M HC1solution, distilled water and pH 7.4 phosphate buffer and then incubated at 37° C. The extracts were filtered with 0.2 μm nylon filter and extracted with 0.5 ml of EtOAc. After adding internal standard, the solvent layers were evaporated with N₂ gas and injected on LC-MS. After 10 minutes, the dissolved percentage of EETs from enteric coated particles in pH 7.4 buffer was almost 100%. In contrast, only a small amount (blew 0.01%) of released EETs were found in the acidic and water solutions. These results suggested release of EETs from the enteric coated particles can be delayed until reaching the duodenum, where solubilization of enteric polymer occurs.

Animals.

Healthy, 11-week-old male spontaneously hypertensive (“SH”) (SHR/NCrlBR) rats (derived from WKY rats by phenotypic segregation of the hypertensive trait and inbreeding) were purchased from Charles River Laboratories (Portage, Mich.) and quarantined for 1 week prior to exposure to tobacco smoke. Animals were handled in accordance with standards established by the U.S. Animal Welfare Acts set forth in National Institutes of Health guidelines and the University of California, Davis Animal Care and Use Committee. The rats were housed in plastic cages with TEK-Chip pelleted paper bedding (Harlan Teklad, Madison, Wis.) and maintained on a 12 hour light/12 hour dark cycle. All animals had access to water and Laboratory Rodent Diet 5001 purchased from LabDiet (Brentwood, Mo.) ad libitum before, during and after exposures.

Treatment of Animals for Pharmacokinetics Study.

Animals were selected for pharmacokinetics studies based on a body-weight stratified randomization procedure after 1-2 weeks acclimation period. The body weight of animals was 250 to 280 g. A 10 mg/kg bodyweight dosing of these inhibitors (7 mg/1 ml corn oil) were subcutaneously administered to SH rats.

Blood sample preparation.

After administration, serial tail bled blood samples (<10 μL) were collected at various time points (30 min to 72 hr). Blood sample was transferred to a 1.5 mL Eppendorf microcentrifuge tube. The blood samples were weighted with analytical balance and vortexed with 100 μL of purified water and 25 μL of internal standard (500 ng/mL CTU). The samples were extracted with 500 μL of ethyl acetate. An ethyl acetate layer was transferred to a 1.5 mL Eppendorf microcentrifuge tube, then dried under nitrogen. The residues were reconstituted in 25 μL of methanol. Aliquots (10 μL) were injected onto LC-MS-MS system.

Pharmacokinetics analysis.

The pharmacokinetic parameters were obtained by fitting the blood concentration-time data to noncompartmental model with the WinNonlin software (Pharsight, Mountain View, Calif.). Parameters estimated included the lambda z (λz), the time of maximum concentration (Tₘₐₓ), the maximum concentration (Cₘₐₓ), elimination half-life (T_{1/2}), area under the concentration-time curve to terminal time (AUCₜ), area under the concentration-time curve to infinite time (AUC_{∞}) and the mean residence time (MRT). AUCₜ was calculated by the linear/log trapezoidal rule.

Synthesis of EETs.

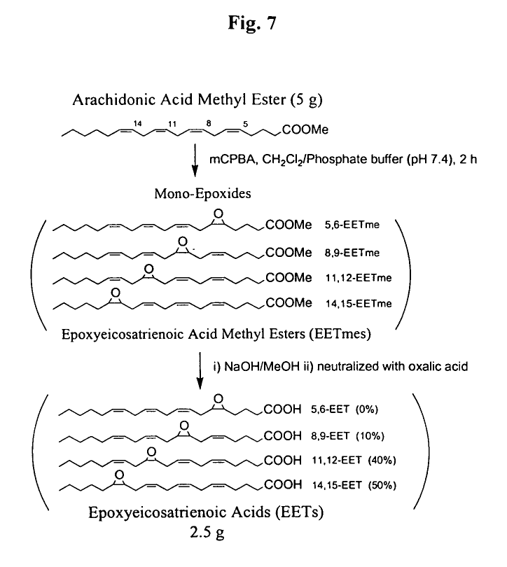

Arachidonic acid methylester (5 g, 16.4 mmol) was epoxidized with m-chloro-perbenzoic acid (mCPBA, 4.3 g, 16.4 mmol) at room temperature in CH₂Cl₂/phosphate buffer (pH 7.4) biphasic system for 2hr. The organic phase was isolated, treated with anhydrous potassium fluoride and filtrated to remove precipitated residual mCPBA. The organic layer was evaporated evaporated in vacuo. The residue was purified by flash chromatography (stepwise elution with hexane-EtOAc: 2, 3, 4 and 6% EtOAc). To a solution of the isolated mono-epoxides in methanol, were added base at 0° C. and incubated 24 hr at room temperature. The mixture was neutralized with oxalic acid and extracted with EtOAC. The organic phase was washed with sat. aq. NaCl and applied to flash chromatography (elution with 10% EtOAc in hexane) to afford the epoxyeicosastrienoic acid mixtures (EETs, 2.5 g, total yield from arachidonic acid methylester: 48% ) of each regio-isomer (10% of 8,9-, 40% of 11,12- and 50% of 14,15-EET). The synthesis is shown in FIG. 7.

LC-MS of EETs.

All of the EETs and each isomer were analyzed by LC-MS as follows. An ESI mass spectrometer was operated in the negative ion mode with a capillary voltage at 1.0 kV. Cone gas (N2) and desolvation gas (N2) were maintained at flow rates of 125 and 643 L/h, respectively. The source and the desolvation temperature were set at 125 ° C. and 400° C., respectively. The optimum cone voltages were set at 55 V. Ultra pure argon (99.9999%) was used as a collision gas at a pressure of 2.5 milli-torr for collision-induced dissociation (CID). Chromatographic separation was performed using a two-solvent linear gradient system. Solvents A and B used were 0.1% acetic acid and 85:15 of acetonitrile:methanol containing 0.1% formic acid, respectively. Solvents were filtered through 0.45 μm membrane and degassed before use. Mobile phases were mixed with a linear gradient from 15% B to 30% B over 0-2 min, 30% B to 55% B over 2-8 min, 55% B to 75% B over 8-28 min, then isocratic for 5 min with 100% B. The post-run was carried out to equilibrate the column to the initial conditions for 1 min before next run. Ten microliters of standard and the extracts were injected onto the column.

Subcutaneous Implantation of EETs for Tobacco Smoke Exposures.

Wax formulations containing EETs were implanted subcutaneously 1 day prior to onset of exposure to tobacco smoke. Animals were implanted with the EETs formulation on the day before the first day of exposure. Four animals from the control group and four animals from the tobacco smoke-exposed group were implanted with the EETs formulation. The approach of a single subcutaneous implantation for the 3-day study was selected to minimize stress to animals from anesthesia.

Subcutaneous injection of AUDA-nBE for Tobacco Smoke Exposures.

AUDA-nBE (7 mg/l ml corn oil) was subcutaneously administered in SH rats at a dose of 10 mg/kg bodyweight. The total volume injected was 0.36 to 0.46 ml. Animals were injected with AUDA-NBE each day prior to exposure. Doses of AUDA-NBE used in this study were selected based on results from preliminary pharmacokinetic studies in mice and rats. These doses were selected to provide optimal efficacy and minimal toxicity over a three-day period. Four animals from the control group and four animals from the tobacco smoke-exposed group were injected with AUDA-NBE. Four animals with EETs implants from the control group and four animals with EETs implants from the tobacco smoke-exposed group were injected with AUDA-nBE. In addition, four control animals and four tobacco smoke-exposed animals were injected with corn oil using the same protocol as the AUDA-nBE-injected animals.

Tobacco Smoke Exposure.

Rats were exposed to a mixture of sidestream and mainstream cigarette smoke in a smoking apparatus (Teague, S. V. et al., Inhal. Toxicol. 6:79-93 (1994)). The cigarettes were humidified 2R4F research cigarettes (Tobacco Health Research Institute; Lexington, Ky.). An automatic metered puffer was used to smoke the cigarettes under Federal Trade Commission conditions (35 ml puff, 2 sec duration, 1 puff per min). The smoke was collected in a chimney, diluted with filtered air, and delivered to whole-body exposure chambers. The exposures were characterized for three major constituents of cigarette smoke; nicotine, carbon monoxide, and total suspended particulates (TSP). Animals were exposed for 6 hours/day for 2 or 15 days. Carbon monoxide was measured every 30 minutes, TSP every 2 hours, and nicotine once per day (approximately midway through the exposure period).

Bronchoalveolar Lavage.

Established protocols were followed for bronchoalveolar lavage (BAL) of animals (Gossart, S. et al., J. Immunol. 156:1540-1548 (1996)). Eighteen hours after the last exposure to tobacco smoke, animals were anesthetized with an overdose of sodium pentobarbital. The trachea was cannulated and the lung lavaged with one aliquot of Ca²+/Mg²+-free phosphate buffered saline (PBS, pH 7.4). The volume of the aliquot was equal to 35 ml/kg body weight (approximately 90% of total lung capacity). The aliquot was instilled into the lungs three times before final collection. The BAL fluid (BALF) was immediately centrifuged at 250×g for 10 min at 4° C. to remove cells. The cell pellet was then resuspended in PBS and the cells counted with a hemocytometer. Cell differentials were performed on cytospin preparations (Shandon, Pittsburgh, Pa.) stained with HEMA 3 (Fisher Scientific, Swedesboro, N.J.). Macrophages, neutrophils, and lymphocytes were counted using light microscopy (1000 cells per sample).

Data Analysis.

All numerical data were calculated as mean±SD or SE. Comparisons between tobacco smoke-exposed and filtered air-exposed controls were made by analysis of variance followed by Fisher's protected least significant difference posttest. A p value of 0.05 or less was considered significant. Statistical analysis was performed with StatView 5.0.1 (SAS Institute Inc., Cary, N.C.).

### Example 2

Tobacco smoke Exposure Characteristics.

TSP, nicotine, and carbon monoxide levels in the tobacco smoke during the 3 day study are shown in Table 1.

Pharmacokinetics.

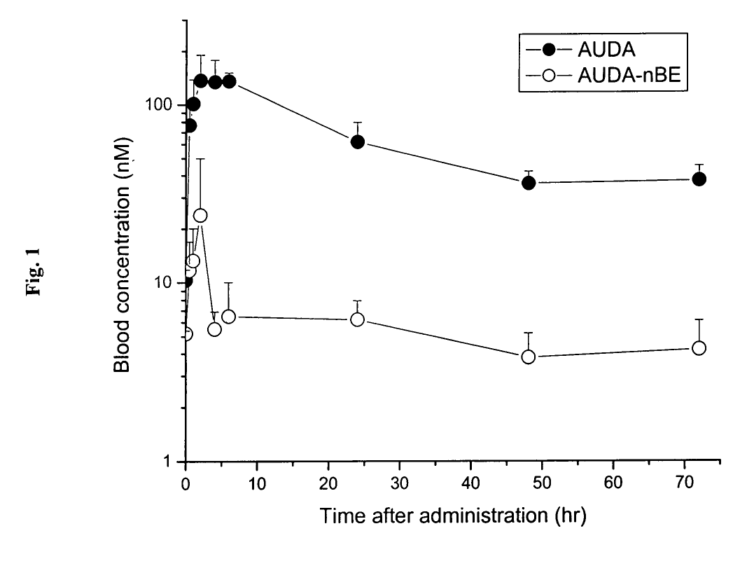

To estimate blood concentration of AUDA-NBE and AUDA in SH rats, pharmacokinetic study was performed with single dose. FIG. 1 shows blood concentration-time profiles of AUDA-NBE and AUDA in SH rats following subcutaneous administration. AUDA-nBE was metabolized to AUDA, which was a potent inhibitor of sEH. Thus, AUDA-NBE was administered as a prodrug for AUDA to improve bioavailability. The half-life of AUDA was 22 hr.

BAL.

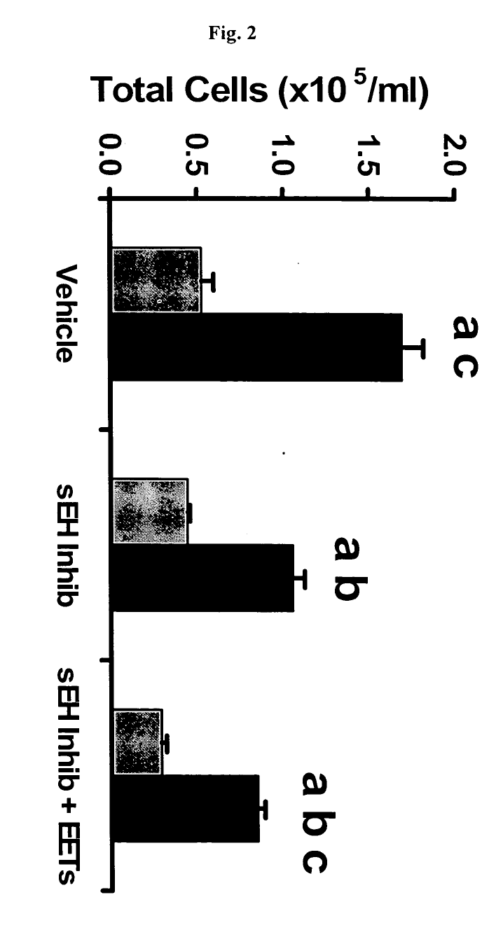

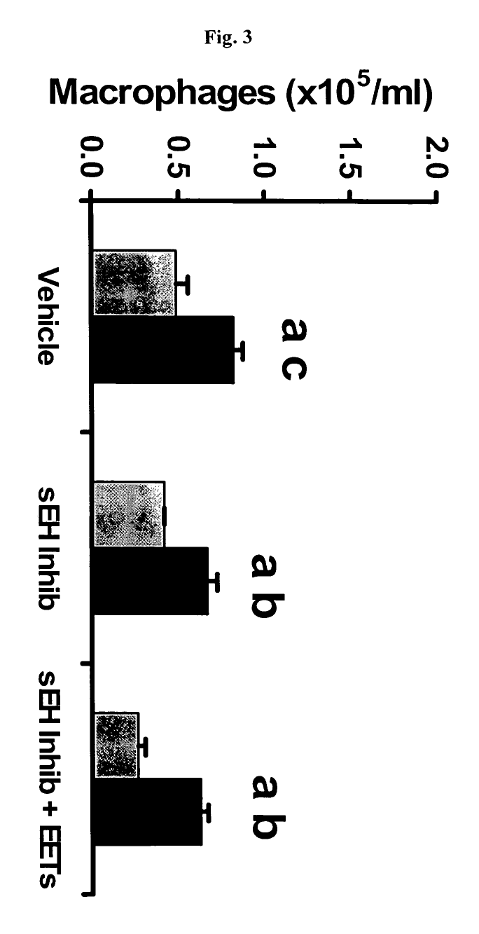

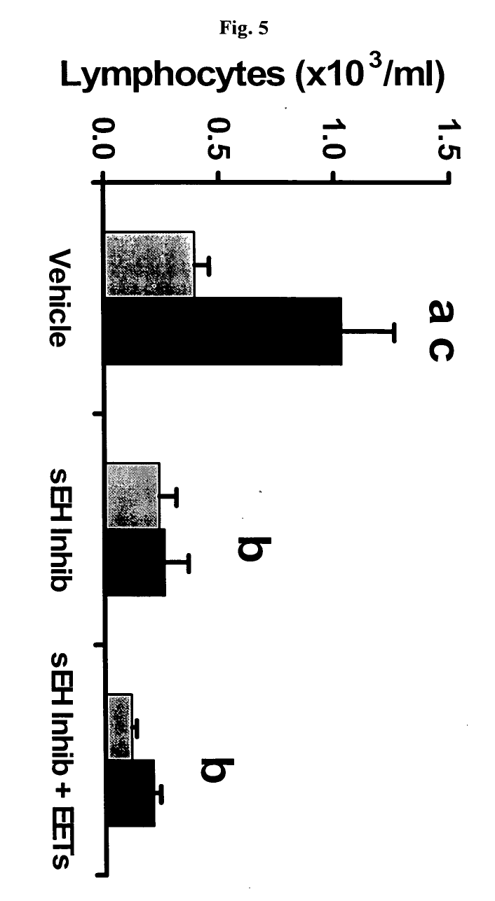

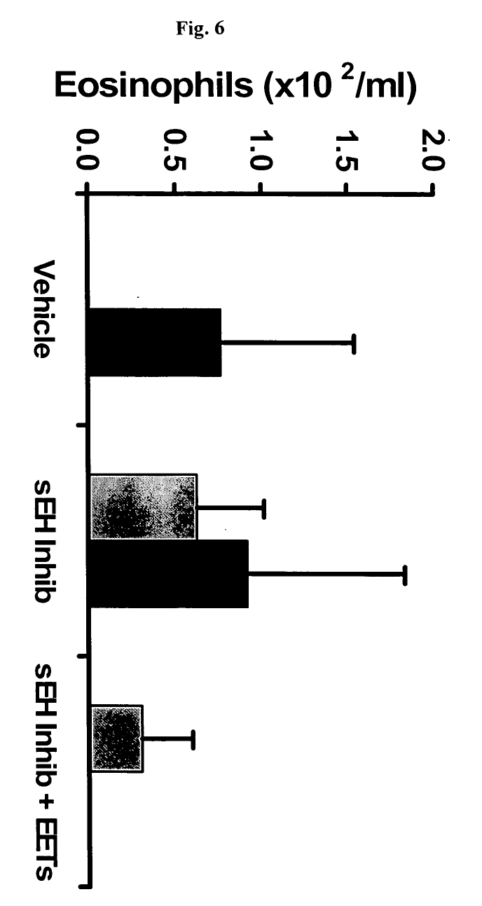

Total number of cells in the BALF was increased significantly after 3 days of tobacco smoke exposure. Subcutaneous injection of AUDA-nBE before exposure significantly decreased the number of BALF cells (FIG. 2). Treatment of animals with both AUDA-NBE and EETs before exposure to tobacco smoke for 3 days resulted in further decrease in total BALF cells compared to treatment with AUDA-NBE alone (FIG. 2). The number of BALF macrophages was increased significantly after either 3 days of tobacco smoke exposure (FIG. 3). Injection of AUDA-NBE prior to exposure significantly decreased the number of BALF macrophages present following 3 days of exposure. A further decrease in number of BALF macrophages recovered was not observed when animals were treated with EETS in addition to AUDA-NBE (FIG. 3). The number of neutrophils in BALF was also significantly increased after 3 days of tobacco smoke exposure (FIG. 4). Injection of AUDA-nBE before exposure significantly decreased the number of BALF neutrophils following 3 days of exposure. Treatment with a combination of AUDA-nBE and EETs before exposure to tobacco smoke resulted in enhanced attenuation of neutrophils recovered by lavage compared to treatment with only AUDA-nBE. Lymphocyte number was also significantly increased in BALF following exposure to tobacco smoke for 3 days (FIG. 5). Injection of AUDA-nBE prior to exposure decreased the number of BALF lymphocytes to levels not significantly different from filtered air controls. Combined treatment of animals with AUDA-NBE and EETs before exposure to tobacco smoke did not result in further reduction of numbers of BAL neutrophils compared to treatment with only AUDA-nBE. Numbers of eosinophils were increased in the BALF following 3 days of tobacco smoke exposure, though not to a statistically significant level (FIG. 6). The cell differentials shown in Table 2 exhibit similar trends to the numbers of different cell types in BALF following exposure to tobacco smoke with or without injection of AUDA-nBE prior to exposure.

### Example 3

Pulmonary inflammation was induced and persisted in rats exposed to tobacco smoke at an average concentration (mean±S.D.) of 76.4±16.0 mg TSP/m³ for 3 days. Subcutaneous injection of AUDA-nBE prior to exposure to tobacco smoke significantly decreased the number of cells in BALF recovered from tobacco smoke-treated rats associated with significant reductions in macrophages, neutrophils, and lymphocytes. The combination of sEH inhibitor and EETs further reduced TS-induced inflammation compared with sEH inhibitor alone.

There is a considerable amount of research to support a key role for inflammation as a driving force to cause the airway epithelium to undergo changes leading to the loss of ciliated cells, hypersecretion of mucin, bronchitis, emphysema, and lung cancer. Smoking causes a local cytokine secretion in the lung, which leads to an infiltration of leukocytes into the airways and alveolar destruction. Reactive oxygen species (ROS) have been shown to play an important role in numerous forms of inflammation (Rahman, I. et al., Free Radic. Biol. Med. 28:1405-1420 (2000); Driscoll, K. E. Toxicol. Lett. 112-113:177-183 (2000); Salvemini, D. et al., Eur. J. Pharmacol. 303:217-220 (1996); Cuzzocrea, S. et al., Free Radic. Biol. Med. 24:450-459 (1998)). The gas and tar phases of tobacco smoke contain oxidants and free radicals (Pryor, W. A. et al., Environ Health Perspect 47:345-355 (1983)) that may cause the sequestration of neutrophils from the pulmonary microcirculation as well as an accumulation of macrophages in respiratory bronchioles (Drost, E. M. et al., Am. J. Respir. Cell Mol. Biol. 6:287-295 (1992)). In addition, alveolar macrophages and neutrophils have the potential to produce large amounts of reactive oxygen intermediates through NADPH oxidase (Emmendorffer, A. et al., J. Immunol. Methods 131:269-275 (1990); Emmendorffer, A. et al., Cytometry 18:147-155 (1994)). Oxidants, either inhaled or generated by inflammatory cells, have been implicated in the inflammatory process in the lungs. A catalytic antioxidant, AEOL 10150, has previously been shown to decrease tobacco smoke-induced inflammation in the lungs of rats, suggesting a role of oxygen radicals in the induction of proinflammatory cytokines and chemokines (Smith, K. R., Free Radic Biol Med 33:1106-1114 (2002)), possibly through the oxidant mediated activation of the redox-sensitive transcription factor, nuclear factor (NF)-κB. However, inflammation induced by tobacco smoke was not resolved to baseline levels by treatment with the antioxidant, suggesting a role of additional mediators of inflammation.

Corticosteroids have anti-inflammatory properties, including inhibition of cytokine secretion, making these compounds useful in treatment of COPD. However, a review of several important studies does not show evidence of significant improvement in symptoms of patients with COPD treated with systemic corticosteroids (Wood-Baker, R. Am J Respir Med 2:451-458 (2003)). This suggests a need for additional treatment modalities for inflammation commonly associated with the onset of COPD.

EETs have a broad spectrum of anti-inflammatory activity and likely act by a mechanism that mediates the effects of several cytokines and promotes expression of several cell adhesion molecules (CAMs) including E-selectin, vascular cell adhesion molecule 1 (VCAM-1), and intercellular adhesion molecule 1 (ICAM-1) (Campbell, W. B. Trends Pharmacol Sci 21:125-127 (2000)). Cytokines produced by leukocytes and macrophages including tumor necrosis factor α(TNF-α) and interleukin 1α(IL-1α) promote expression of CAMs. EETs are potent inhibitors of CAM expression induced by TNF-α and IL-1α, with the effect on VCAM-1 the most pronounced (Node, K. et al., Science 285:1276-1279 (1999)). Cytokines induce expression of CAMs, and other inflammatory proteins though activation of the nuclear transcription factor κB (NF-κB). In its inactive form, NF-κB is bound to an inhibitory protein IκB in the cytoplasm (Karin, M. J Biol Chem 274:27339-27342 (1999)). TNF-α and IL-1activate IκB kinase, which phosphorylates critical serines on IκB and results in degradation of the protein. The free subunits of NF-KB are translocated from the cytoplasm to the nucleus where they bind genes encoding pro-inflammatory CAMs resulting in their transcription. EETs act by inhibiting both degradation of IKB and NF-KB-mediated gene transcription (Node, K. et al., Science 285:1276-1279 (1999)).

The enzyme involved in clearance of EETs, (Zeldin, D. C. et al., J Biol Chem 268:6402-6407 (1993)) sEH, may have an important role in regulating EET levels and may therefore be an important mediator of inflammation in the lung. sEH functions in vivo to metabolize EETs to their corresponding dihydroxy derivatives (Fang, X. et al., J Biol Chem 276:14867-14874 (2001)). This enzyme has over 90% homology between rodent and human (Arand, M. et al., FEBS Lett 338:251-256 (1994)) and can be inhibited in vitro by a number of urea, carbamate, and amide derivatives (Morisseau, C. et al., Proc Natl Acad Sci USA 96:8849-8854 (1999); Morisseau, C. et al., Biochem Pharmacol 63:1599-1608 (2002)). Injection of one such inhibitor N,N′-dicyclohexyl urea (DCU) in SH rats resulted in lower blood pressure, an increase in urinary 14,15-EET, and a decrease in urinary dihydroxy derivative. These observations are consistent with in vivo inhibition of sEH by DCU.

TABLE 1
Tobacco smoke characteristics^{a}

| Total suspended | | |
|---|---|---|
| particulate | Nicotine | Carbon |
| (mg/m³) | (mg/m³) | monoxide (ppm) |
| 76.4 ± 16.0 | 6.8 | 234 ± 2 |

^{a}Data are presented as Mean ± SD.

TABLE 2
Cell differentials in BAL after 3 days of tobacco smoke exposure in rats*

| | Vehicle (corn oil) | | sEH Inhibitor | | sEH Inhibitor + EETs | |
|---|---|---|---|---|---|---|
| | Filtered | Tobacco | Filtered | Tobacco | Filtered | Tobacco |
| | Air | Smoke | Air | Smoke | Air | Smoke |
| % | 90.2 ± 1.5 | 48.7 ± 3.4^{†§} | 92.1 ± 1.6 | 62.9 ± 1.8^{†‡} | 93.4 ± 1.9 | 72.7 ± 3.4^{†‡§} |
| Macrophages | | | | | | |
| % Neutrophils | 9.0 ± 1.3 | 50.7 ± 3.4^{†§} | 7.3 ± 1.8 | 36.8 ± 1.6^{†‡} | 6.2 ± 1.9 | 27.1 ± 3.4^{†‡§} |
| % | 0.80 ± 0.16 | 0.60 ± 0.12^{§} | 0.55 ± 0.15 | 0.25 ± 0.10^{‡} | 0.40 ± 0.08 | 0.25 ± 0.05^{‡} |
| Lymphocytes | | | | | | |
| % Eosinophils | 0.00 ± 0.00 | 0.05 ± 0.05 | 0.15 ± 0.10 | 0.10 ± 0.10 | 0.10 ± 0.10 | 0.00 ± 0.00 |

*Data are presented as mean ± SE (n = 4).
^{†}p < 0.05, compared to respective filtered air control.
^{‡}p < 0.05, compared to tobacco smoke + vehicle.
^{§}p < 0.05, compared to tobacco smoke + sEH inhibitor.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

1. A use of a cis-epoxyeicosantrienoic acid (“EET”) for the manufacture of a medicament to inhibit or slow progression of a condition selected from the group consisting of an obstructive pulmonary disease, an interstitial lung disease, and asthma.

2. A use of claim 1, wherein said obstructive pulmonary disease is selected from the group consisting of chronic obstructive pulmonary disease (“COPD”), emphysema, and chronic bronchitis.

3. A use of claim 1, wherein the interstitial lung disease is idiopathic pulmonary fibrosis.

4. A use of claim 1, wherein the interstitial lung disease is one associated with occupational exposure to a dust.

5. A use of claim 1, wherein the condition is asthma.

6. A use of claim 1, wherein said EET is selected from the group consisting of 14,15-EET, 8,9-EET and 1 1,12-EET.

7. A use of claim 1, wherein said EET is 14R,15S-EET.

8. A use of claim 1, wherein the EET is in a material which releases the EET into the surrounding environment over time.

9. A use of an inhibitor of soluble epoxide hydrolase (“sEH”) for the manufacture of a medicament to inhibit or slow progression a condition selected from the group consisting of an obstructive pulmonary disease, an interstitial lung disease, and asthma.

10. A use of claim 9, wherein the obstructive pulmonary disease is selected from the group consisting of chronic obstructive pulmonary disease (“COPD”), emphysema, and chronic bronchitis.

11. A use of claim 9, wherein the interstitial lung disease is idiopathic pulmonary fibrosis.

12. A use of claim 9, wherein the interstitial lung disease is one associated with occupational exposure to a dust.

13. A use of claim 9, wherein the condition is asthma.

14. A use of claim 9, wherein said inhibitor of sEH is selected from the group consisting of an adamantyl dodecyl urea, N-cyclohexyl-N′-dodecyl urea (CDU) and N, N′-dicyclohexylurea (DCU).

15. A use of claim 9, wherein the medicament is a slow release formulation.

16. A use of claim 9, wherein said medicament further comprises a cis-epoxyeicosantrienoic acid (“EET”).

17. A use of claim 9, wherein said EET is selected from the group consisting of 14,15-EET, 8,9-EET and 11,12-EET.

18. A use of claim 9, wherein said EET is 14R,15S-EET.

19. A use of a nucleic acid that inhibits expression of soluble epoxide hydrolase (“sEH”) for the manufacture of a medicament for inhibiting or slowing progression of a condition selected from the group consisting of an obstructive pulmonary disease, an interstitial lung disease, and asthma.

20. A use of claim 19, wherein the nucleic acid is a small interfering RNA.

21. A use of claim 19, wherein said obstructive pulmonary disease is selected from the group consisting of chronic obstructive pulmonary disease (“COPD”), emphysema, and chronic bronchitis.

22. A use of claim 19, wherein the interstitial lung disease is idiopathic pulmonary fibrosis.

23. A use of claim 19, wherein the interstitial lung disease is one associated with occupational exposure to a dust.

24. A use of claim 19, wherein the condition is asthma.

25. A method of inhibiting progression of a condition selected from the group consisting of an obstructive pulmonary disease, an interstitial lung disease, and asthma, said method comprising administering an inhibitor of soluble epoxide hydrolase (“sEH”) and a cis-epoxyeicosantrienoic acid (“EET”) to a person in need thereof.

26. A method of claim 25, wherein said obstructive pulmonary disease is selected from the group consisting of chronic obstructive pulmonary disease (“COPD”), emphysema, and chronic bronchitis.

27. A method of claim 25, wherein the interstitial lung disease is idiopathic pulmonary fibrosis.

28. A method of claim 25, wherein the interstitial lung disease is one associated with occupational exposure to a dust.

29. A method of claim 25, wherein the condition is asthma.

30. A method of claim 25, wherein the inhibitor of sEH or the EET, or both, is in a material which releases the inhibitor over time.

31. A method of claim 25, wherein said EET is selected from the group consisting of 14,15-EET, 8,9-EET and 1 1,12-EET.

32. A method of claim 25, wherein said EET is 14R,15S-EET.

33. A method of claim 25, wherein the inhibitor is administered orally.

34. A method of claim 25, wherein the inhibitor is administered in a total daily dose from about 0.001 mg/kg to about 100 mg/kg body weight.

35. A method of inhibiting progression of a condition selected from the group consisting of an obstructive pulmonary disease, an interstitial lung disease, and asthma, said method comprising administering to a person in need thereof a nucleic acid which inhibits expression of a gene encoding soluble epoxide hydrolase (“sEH”), and a cis-epoxyeicosantrienoic acid (“EET”).

36. A method of claim 35, wherein the obstructive pulmonary disease is selected from the group consisting of chronic obstructive pulmonary disease (“COPD”), emphysema, and chronic bronchitis.

37. A method of claim 35, wherein the interstitial lung disease is idiopathic pulmonary fibrosis.

38. A method of claim 35, wherein the interstitial lung disease is one associated with occupational exposure to a dust.

39. A method of claim 35, wherein the condition is asthma.

40. A method of claim 35, wherein the nucleic acid is a small interfering RNA (“siRNA”).


SEQUENCE LISTING (38 sequences)

SEQ ID NO: 1-2 - skipped (2 entries)

SEQ ID NO: 3 - DNA, 23 bp, Artificial Sequence
  Description of Artificial Sequence:soluble epoxide hydrolase target sequence
cagtgttcattggccatgactgg

SEQ ID NO: 4 - DNA, 21 bp, Artificial Sequence
  Description of Combined DNA/RNA Molecule: soluble epoxide hydrolase sense-siRNA
  Description of Artificial Sequence:soluble epoxide hydrolase sense-siRNA
guguucauuggccaugacutt

SEQ ID NO: 5 - DNA, 21 bp, Artificial Sequence
  Description of Combined DNA/RNA Molecule: soluble epoxide hydrolase antisense-siRNA
  Description of Artificial Sequence:soluble epoxide hydrolase antisense-siRNA
agucauggccaaugaacactt

SEQ ID NO: 6 - DNA, 23 bp, Artificial Sequence
  Description of Artificial Sequence:soluble epoxide hydrolase target sequence
gaaaggctatggagagtcatctg

SEQ ID NO: 7 - DNA, 21 bp, Artificial Sequence
  Description of Combined DNA/RNA Molecule: soluble epoxide hydrolase sense-siRNA
  Description of Artificial Sequence:soluble epoxide hydrolase sense-siRNA
aaggcuauggagagucauctt

SEQ ID NO: 8 - DNA, 21 bp, Artificial Sequence
  Description of Combined DNA/RNA Molecule: soluble epoxide hydrolase antisense-siRNA
  Description of Artificial Sequence:soluble epoxide hydrolase antisense-siRNA
gaugacucuccauagccuutt

SEQ ID NO: 9 - DNA, 23 bp, Artificial Sequence
  Description of Artificial Sequence:soluble epoxide hydrolase target sequence
aaaggctatggagagtcatctgc

SEQ ID NO: 10 - DNA, 21 bp, Artificial Sequence
  Description of Combined DNA/RNA Molecule: soluble epoxide hydrolase sense-siRNA
  Description of Artificial Sequence:soluble epoxide hydrolase sense-siRNA
aggcuauggagagucaucutt

SEQ ID NO: 11 - DNA, 21 bp, Artificial Sequence
  Description of Combined DNA/RNA Molecule: soluble epoxide hydrolase antisense-siRNA
  Description of Artificial Sequence:soluble epoxide hydrolase antisense-siRNA
agaugacucuccauagccutt

SEQ ID NO: 12 - DNA, 23 bp, Artificial Sequence
  Description of Artificial Sequence:soluble epoxide hydrolase target sequence
caagcagtgttcattggccatga

SEQ ID NO: 13 - DNA, 21 bp, Artificial Sequence
  Description of Combined DNA/RNA Molecule: soluble epoxide hydrolase sense-siRNA
  Description of Artificial Sequence:soluble epoxide hydrolase sense-siRNA
agcaguguucauuggccautt

SEQ ID NO: 14 - DNA, 21 bp, Artificial Sequence
  Description of Combined DNA/RNA Molecule: soluble epoxide hydrolase antisense-siRNA
  Description of Artificial Sequence:soluble epoxide hydrolase antisense-siRNA
auggccaaugaacacugcutt

SEQ ID NO: 15 - DNA, 23 bp, Artificial Sequence
  Description of Artificial Sequence:soluble epoxide hydrolase target sequence
cagcacatggaggactggattcc

SEQ ID NO: 16 - DNA, 21 bp, Artificial Sequence
  Description of Combined DNA/RNA Molecule: soluble epoxide hydrolase sense-siRNA
  Description of Artificial Sequence:soluble epoxide hydrolase sense-siRNA
gcacauggaggacuggauutt

SEQ ID NO: 17 - DNA, 21 bp, Artificial Sequence
  Description of Combined DNA/RNA Molecule: soluble epoxide hydrolase antisense-siRNA
  Description of Artificial Sequence:soluble epoxide hydrolase antisense-siRNA
aauccaguccuccaugugctt

SEQ ID NO: 18 - DNA, 9 bp, Artificial Sequence
  Description of Artificial Sequence:nine- nucleotide spacer, linker
ttcaagaga

SEQ ID NO: 19 - DNA, 23 bp, Artificial Sequence
  Description of Artificial Sequence:hairpin siRNA target sequence
cagtgttcattggccatgactgg

SEQ ID NO: 20 - DNA, 59 bp, Artificial Sequence
  Description of Artificial Sequence:hairpin siRNA sense strand
gatccccgtgttcattggccatgactttcaagagaagtcatggccaatgaacacttttt

SEQ ID NO: 21 - DNA, 59 bp, Artificial Sequence
  Description of Artificial Sequence:hairpin siRNA antisense strand
agctaaaaagtgttcattggccatgacttctcttgaaagtcatggccaatgaacacggg

SEQ ID NO: 22 - DNA, 23 bp, Artificial Sequence
  Description of Artificial Sequence:hairpin siRNA target sequence
gaaaggctatggagagtcatctg

SEQ ID NO: 23 - DNA, 59 bp, Artificial Sequence
  Description of Artificial Sequence:hairpin siRNA sense strand
gatccccaaggctatggagagtcatcttcaagagagatgactctccatagccttttttt

SEQ ID NO: 24 - DNA, 59 bp, Artificial Sequence
  Description of Artificial Sequence:hairpin siRNA antisense strand
agctaaaaaaaggctatggagagtcatctctcttgaagatgactctccatagccttggg

SEQ ID NO: 25 - DNA, 23 bp, Artificial Sequence
  Description of Artificial Sequence:hairpin siRNA target sequence
aaaggctatggagagtcatctgc

SEQ ID NO: 26 - DNA, 59 bp, Artificial Sequence
  Description of Artificial Sequence:hairpin siRNA sense strand
gatccccaggctatggagagtcatctttcaagagaagatgactctccatagcctttttt

SEQ ID NO: 27 - DNA, 59 bp, Artificial Sequence
  Description of Artificial Sequence:hairpin siRNA antisense strand
agctaaaaaaggctatggagagtcatcatctcttgaaagatgactctccatagcctggg

SEQ ID NO: 28 - DNA, 23 bp, Artificial Sequence
  Description of Artificial Sequence:hairpin siRNA target sequence
caagcagtgttcattggccatga

SEQ ID NO: 29 - DNA, 59 bp, Artificial Sequence
  Description of Artificial Sequence:hairpin siRNA sense strand
gatccccagcagtgttcattggccatttcaagagaatggccaatgaacactgctttttt

SEQ ID NO: 30 - DNA, 59 bp, Artificial Sequence
  Description of Artificial Sequence:hairpin siRNA antisense strand
agctaaaaaagcagtgttcattggccattctcttgaaatggccaatgaacactgctggg

SEQ ID NO: 31 - DNA, 23 bp, Artificial Sequence
  Description of Artificial Sequence:hairpin siRNA target sequence
cagcacatggaggactggattcc

SEQ ID NO: 32 - DNA, 59 bp, Artificial Sequence
  Description of Artificial Sequence:hairpin siRNA sense strand
gatccccgcacatggaggactggattttcaagagaaatccagtcctccatgtgcttttt

SEQ ID NO: 33 - DNA, 59 bp, Artificial Sequence
  Description of Artificial Sequence:hairpin siRNA antisense strand
agctaaaaagcacatggaggactggatttctcttgaaaatccagtcctccatgtgcggg

SEQ ID NO: 34 - RNA, 20 nt, Artificial Sequence
  Description of Artificial Sequence:soluble epoxide hydrolase exemplary antisense sequence
uguccagugcccacaguccu

SEQ ID NO: 35 - RNA, 20 nt, Artificial Sequence
  Description of Artificial Sequence:soluble epoxide hydrolase exemplary antisense sequence
uucccaccugacacgacucu

SEQ ID NO: 36 - RNA, 20 nt, Artificial Sequence
  Description of Artificial Sequence:soluble epoxide hydrolase exemplary antisense sequence
guucagccucagccacuccu

SEQ ID NO: 37 - RNA, 19 nt, Artificial Sequence
  Description of Artificial Sequence:soluble epoxide hydrolase exemplary antisense sequence
aguccucccgcuucacaga

SEQ ID NO: 38 - RNA, 21 nt, Artificial Sequence
  Description of Artificial Sequence:soluble epoxide hydrolase exemplary antisense sequence
gcccacuuccaguuccuuucc